(19) United States
(12) Patent Application Publication
(10) Pub. No.: US 2009/0191597 A1
(43) Pub. Date: Jul. 30, 2009

(54) ENHANCED PRODUCTION OF INFECTIOUS PARVOVIRUS VECTORS IN INSECT CELLS

(75) Inventors: Jude Samulski, Chapel Hill, NC (US); Haifeng Chen, Castro Valley, CA (US)

Correspondence Address: MOORE & VAN ALLEN PLLC, P.O. BOX 13706, Research Triangle Park, NC, 27709, US

(73) Assignee: ASKLEPIOS BIOPHARMACEUTICAL, INC., Chapel Hill, NC (US)

(21) Appl. No.: 12/161,202

(22) PCT Filed: Jan. 22, 2007
(86) PCT No.: PCT/US2007/001668

§ 371 Date: Dec. 9, 2008

Related U.S. Application Data
(60) Provisional application No. 60/760,812, filed on Jan. 20, 2006.
(60) Provisional application No. 60/765,665, filed on Feb. 6, 2006.
(60) Provisional application No. 60/804,772, filed on Jun. 14, 2006.

(51) Int. Cl. C12P 19/34; C12N 5/06

(52) U.S. Cl. 435/91.4; 435/325

(57) ABSTRACT

A method of producing a packaged parvovirus vector, the method comprising: (a) providing an insect cell; (b) introducing into the insect cell one or more vectors comprising nucleotide sequences encoding: (i) a transgene flanked by TRs; and (ii) baculovirus packaging functions comprising Rep components and Cap components sufficient to result in packaging of infective parvovirus particles, wherein VP1 is supplemented relative to VP2 and VP3 sufficient to increase the production of infectious viral particles; and (c) introducing into the cell a nucleic acid encoding baculovirus helper functions for expression in the insect cell; (d) culturing the cell under conditions sufficient to produce the infectious packaged parvovirus vector.

### CROSS REFERENCE TO RELATED APPLICATIONS

This application claims priority from U.S. Provisional Patent Application No. 60/760,812, filed on Jan. 20, 2006; U.S. Provisional Patent Application No. 60/765,665 filed on Feb. 6, 2006; and U.S. Provisional Patent Application No. 60/804,772, filed on Jun. 14, 2006, all entitled “ENHANCED PRODUCTION OF INFECTIOUS PARVOVIRUS VECTORS IN INSECT CELLS,” the contents of which are hereby incorporated by reference herein.

### BACKGROUND OF THE INVENTION

1. Field of the Invention

The present invention relates to the production of parvovirus vectors, and more particularly, to the production of recombinant adeno-associated viruses (rAAV) in insect cells and uses thereof.

2. Description of the Related Art

Viruses of the Parvoviridae family are small DNA viruses characterized by, among other things, their ability to infect particular hosts. The family Parvoviridae includes two subfamilies: the Parvovirinae, which infect vertebrates, and the Densovirinae, which infect insects. The subfamily Parvovirinae (referred to as the parvoviruses) includes the genus Dependovirus, the members of which are unique in that, under most conditions, these viruses require coinfection with a helper virus such as adenovirus or herpes virus for productive infection. The genus Dependovirus includes adeno-associated virus (AAV), which normally infects humans (e.g., serotypes 2, 3A, 3B, 5, and 6) or primates (e.g., serotypes 1 and 4), and related viruses that infect other warm-blooded animals (e.g., bovine, canine, equine, and ovine adeno-associated viruses). The parvoviruses and other members of the Parvoviridae family are generally described in Kenneth I. Berns, “Parvoviridae: The Viruses and Their Replication,” Chapter 69 in FIELDS VIROLOGY (3d Ed. 1996).

In recent years, AAV has emerged as a preferred viral vector for gene therapy due to its ability to efficiently infect both nondividing and dividing cells, integrate into a single chromosomal site in the human genome, and pose relatively low pathogenic risk to humans. In view of these advantages, recombinant adeno-associated virus (rAAV) presently is being used in gene therapy clinical trials for hemophilia B, malignant melanoma, cystic fibrosis, and other diseases.

The difficulties involved in scaling-up rAAV production for clinical trials and commercialization using current mammalian cell production systems can be significant, if not entirely prohibitive. For example, for certain clinical studies more than 1015 particles of rAAV may be required. To produce this number of rAAV particles, transfection and culture with approximately 1011 cultured human 293 cells, the equivalent of 5,000 175-cm² flasks of cells, would be required. Related difficulties associated with the production of AAV using known mammalian cell lines are recognized in the art. There also is the possibility that a vector destined for clinical use produced in a mammalian cell culture will be contaminated with undesirable, perhaps pathogenic, material present in a mammalian cell. To further compound the difficulties of using parvovirus or AAV as vectors, the entire process of engineering a new vector and expressing the desired polypeptides in a stably transfected cell line is a time- and labor-intensive undertaking. Further, recent developments in the use of insect cells to produce AAV have resulted in the production on mostly non-infectious particles.

Thus, there remains a need for improved methods and tools for producing parvoviral vectors. The vector manufacturing systems of the present invention improve the simplicity and efficiency of the process for creating parvoviral vectors. There also remains a need in the art for improved methods of using insect cells in the production of infective AAV particles.

### SUMMARY OF THE INVENTION

The present invention provides for novel methods, host cells and vector constructs which permit the efficient production of infectious rAAV by increasing the expression of the VP1 structural component while leaving the expression of the VP2 and VP3 structural components at an essentially normal level.

In one aspect the present invention provides a method of producing a packaged parvovirus vector. In general, the method of the invention comprises:

(a) providing a host cell;
(b) introducing into the host cell one or more vectors comprising nucleotide sequences encoding:
(i) a transgene flanked by TRs; and
(ii) virus packaging functions comprising parvovirus Rep components and/or parvovirus Cap components sufficient to result in packaging of infective parvovirus particles, wherein VP1 is supplemented relative to VP2 and VP3 sufficient to increase the production of infectious viral particles and under the control of regulatory sequences directing expression in the host cell; and
(c) introducing into the insect cell nucleic acid encoding virus helper functions for expression in the insect cell;
(d) culturing the host cell under conditions sufficient to produce the infectious packaged parvovirus vector.

In another aspect, the present invention provide for a method of rAAV production in insect cells, the method comprising:

(a) providing an insect cell;
(b) introducing into the insect cell one or more baculovirus vectors comprising nucleotide sequences encoding:
(i) a transgene flanked by AAV TRs; and
(ii) baculovirus packaging functions comprising AAV Rep components and AAV Cap components sufficient to result in packaging of infective parvovirus particles, wherein VP1 is supplemented relative to VP2 and VP3 sufficient to increase the production of infectious viral particles;
(c) introducing into the insect cell a nucleic acid encoding baculovirus helper functions for expression in the insect cell; and
(d) culturing the insect cell under conditions sufficient to produce the infectious packaged parvovirus vector.

Preferably, the virus packaging functions are derived from a baculovirus expression system. In one embodiment, the baculovirus packaging system or vectors may be constructed to carry the AAV Rep and Cap coding region by engineering these genes into the polyhedrin coding region of a baculovirus vector and producing viral recombinants by transfection into a host cell. Preferably, the host cell is a baculovirus-infected cell or has introduced therein additional nucleic acid encoding baculovirus helper functions or includes these baculovirus helper functions therein. These baculovirus viruses can express the AAV components and subsequently facilitate the production of the capsids. Host cells may include Sf9 and Sf21.

In preferred embodiments, codon optimization of AAV rep proteins may be undertaken to alter homology, reducing adverse or unanticipated genomic alterations resulting from recombination events between homologous nucleotides. Accordingly, when the host is an insect cell, the sequences encoding the AAV Rep, AAVCap, VP1, VP2, and/or VP3 coding regions may be preferably codon-optimized for expression in the particular host insect cell. Thus, for example, the sequence of AAV2 Rep52 as shown in FIG. 12 (SEQ ID NO. 1) can be optimized for expression in the insect cell as shown in FIG. 13 (SEQ ID NO. 2); the sequence of AAV2 Rep78 as shown in FIG. 14 (SEQ ID NO. 3) can be optimized for expression in the insect cell as shown in FIG. 15 (SEQ ID NO. 4); the sequence of AAV2 Capsid 2.5 as shown in FIG. 16 (SEQ ID NO. 5) can be optimized for expression in the insect cell as shown in FIG. 17 (SEQ ID NO. 6); the sequence of AAV8 VP1 as shown in FIG. 18 (SEQ ID NO. 7) can be optimized for expression in the insect cell as shown in FIG. 19 (SEQ ID NO. 8); the sequence of AAV9 VP1 as shown in FIG. 20 (SEQ ID NO. 9) can be optimized for expression in the insect cell as shown in FIG. 21 (SEQ ID NO. 10); the nucleotide sequence of AAV2 VP1 as shown in FIG. 22 (SEQ ID NO. 11) can be mutated to optimized as shown in FIG. 16 (SEQ ID NO. 5); and the amino acid sequence of AAV2 VP1 as shown in FIG. 23 (SEQ ID NO. 12) can be mutated to be optimized as shown in FIG. 24 (SEQ ID NO. 13). As such, the codons would be optimized for usage in the insect cells. The optimized sequences further include nucleotide sequences having substantial homology.

The present invention further provides a method of delivering a transgene for expression in a cell comprising administering to the cell one or more vectors comprising nucleotide sequences encoding:

(i) a transgene flanked by TRs; and
(ii) baculovirus helper functions for the replication of Rep components and Cap components and packaging of infective parvovirus particles, wherein VP1 is supplemented relative to VP2 and VP3 sufficient to increase the production of infectious viral particles.

The supplementation of VP1 may, for example, be effected by

(a) introducing into the insect cell a Cap vector comprising nucleotide sequences expressing VP1, VP2 and VP3 and introducing into the insect cell a VP1 vector comprising nucleotide sequences expressing VP1; or
(b) introducing into the insect cell a single vector comprising nucleotide sequences for the Cap component (for expression of VP1, VP2 and VP3) and also nucleotide sequences for the VP1 component.

In another aspect, the present invention provides a method of delivering a transgene for expression in a cell comprising administering to the cell one or more baculovirus vectors comprising nucleotide sequences encoding:

(i) a transgene flanked by AAV ITRs; and
(ii) baculovirus helper functions for the replication of AAV Rep components and AAV Cap components included in the vector, wherein VP1 is supplemented relative to VP2 and VP3 sufficient to increase the production of infectious viral particles.

The AAV rep components may inserted in a separate vector from that of the Cap components. Alternatively, the additional VP1 component may be inserted in the same vector as that of the Rep components or the Cap components. The Rep components may be included in separate vectors.

The Cap vector and the VP1 vector can typically be introduced into the insect cell at a moi which is at least 1. The vectors may be introduced into the cell simultaneously or in serial, and preferably simultaneously in an amount sufficient to cause an increase in production of infectious viruses.

In one embodiment, expression control sequences of the VP1 vector provide relatively weaker expression as compared to expression control sequences of the Cap vector. For example, expression of the VP1 vector is suitably from about 1 to about 75% of the expression of the Cap vector, from about 1 to about 50% of the expression of the Cap vector, or from about 1 to about 25% of the expression of the Cap vector. Weaker expression may be provided, for example, using a mutated polyhedrin promoter. Ideally, the supplementation of VP1 results in production of a molecular ratio of approximately 10:10:80 VP1:VP2:VP3. The supplementation of VP1 may result in production of infectious packaged parvovirus vectors in an amount which is about 10, 20, 30, 40, 50, 60, 70, 80, 90, 100, 110, 120, 130, 140, 15, 160, 170, 180, 190 or 200 times greater than in corresponding method in the absence of the supplementation.

Insect cells used in accordance with the method are preferably from the order Lepidoptera or are derived from cells of this order. Preferably, the insect cells are from the genus Spodoptera or Trichopulsia, e.g., Spodoptera frugiperda or Trichopulsia ni. Preferred cell lines include SF9, SF21, High Five™ cells (BRI-TN-5B1-4), Mimic-SF9 cells derived from any of the foregoing.

In yet another aspect, the present invention provides for a recombinant host cell containing at least one vector, wherein the at least one vector comprises nucleotide sequences encoding: a transgene flanked by parvovirus TRs; and

baculovirus packaging functions and parvovirus Rep and Cap components sufficient to result in packaging of infective parvovirus particles, wherein VP1 is supplemented relative to VP2 and VP3 sufficient to increase the production of infectious viral particles.

In a still further aspect, the invention provides for a kit for expressing viral particles, wherein the kit comprises at least two vectors, wherein the vectors comprise nucleotide sequences encoding: a transgene flanked by parvovirus TRs; and

baculovirus packaging functions comprising parvovirus Rep components and Cap components sufficient to result in packaging of infective parvovirus particles, wherein VP1 is supplemented relative to VP2 and VP3 sufficient to increase the production of infectious viral particles.

The kit may further comprise insect cells and a nucleic acid encoding baculovirus helper functions for expression in the insect cell.

### BRIEF DESCRIPTION OF THE FIGURES

FIG. 1 illustrates four recombinant baculoviruses that may be used in AAV production.

FIG. 2 shows the lower VP1/VP3 ratio from vectors produced in insect cells.

FIG. 3 shows the increased VP1 levels in cell lysates by coinfection with VP1 expressing baculovirus.

FIG. 4 shows the increased VP1 levels in viral particles produced in cells infected with VP1 expressing baculovirus.

FIG. 5 shows the results of transduction of HepG2 cells by AAV2.5-GFP vectors produced in Sf9 and 293 cells.

FIG. 6 shows the improvement of AAV-2.5GFP infectivity by adding extra 2.5VP1.

FIG. 7 illustrates four additional recombinant baculoviruses that may be used in AAV production, wherein the vectors illustrate inclusion of terminal repeats (ITR) and promoters.

FIG. 8 illustrates a system for producing infectious AAV wherein the system includes only three vectors, wherein the genes encoding VP1, VP2 and VP3 are include in the same vector with AAV replication components.

FIG. 9 illustrates a system for producing infectious AAV wherein the system includes only two vectors, wherein the wherein the genes encoding VP1, VP2 and VP3 are included in the same vector with AAV replication components, and the second vector includes genes for GFP and the additional gene for increased expression of VP1.

FIG. 10 illustrates a system for producing infectious AAV wherein the system includes only two vectors, wherein a replication component is included in each vector.

FIG. 11 illustrates a system for producing infectious AAV wherein the system includes two vectors, wherein the replication components are included in a single vector and the genes encoding VP1, VP2 and VP3 are included in the same vector as the additional gene for expressing increased levels of VP1.

FIG. 12 shows the nucleotide sequence encoding for AAV2 Rep52 protein (SEQ ID NO. 1).

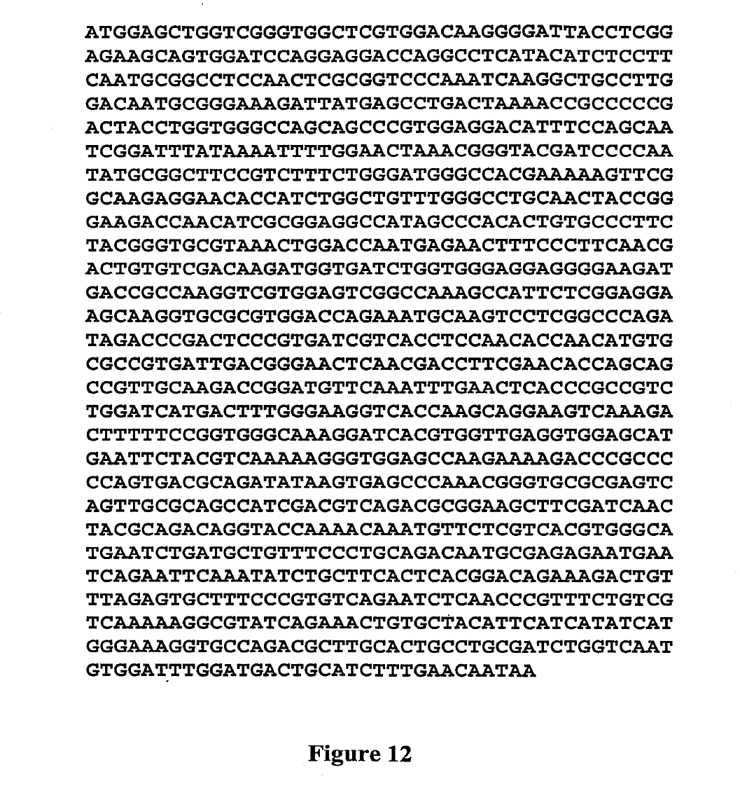

FIG. 13 shows the optimized nucleotide sequence encoding for expression of the AAV2 Rep52 protein in insect cells (SEQ ID NO. 2).

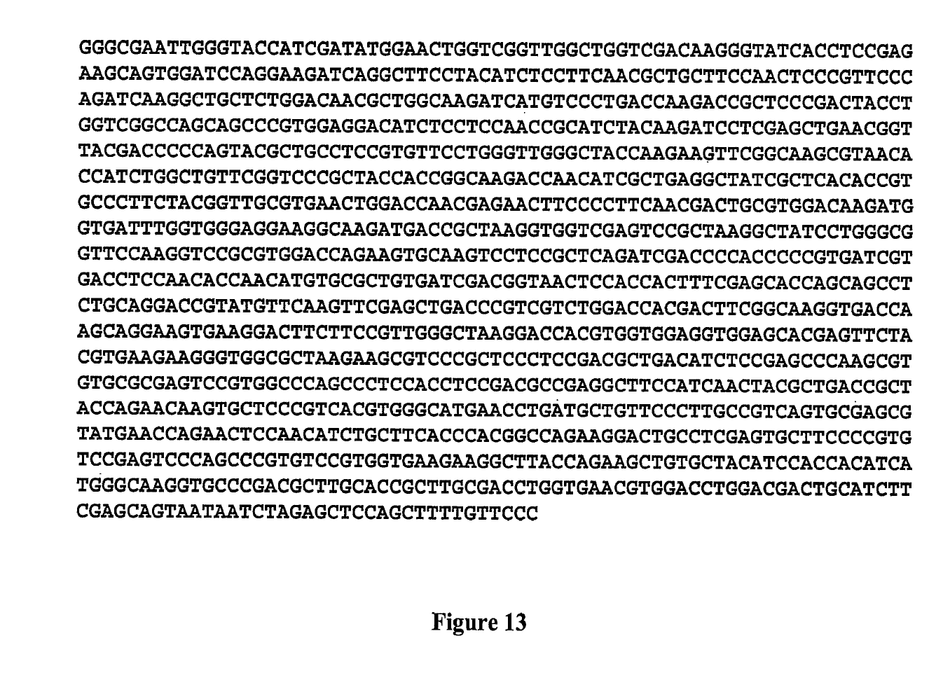

FIG. 14 shows the nucleotide sequence encoding for AAV2 Rep78 protein (SEQ ID NO. 3).

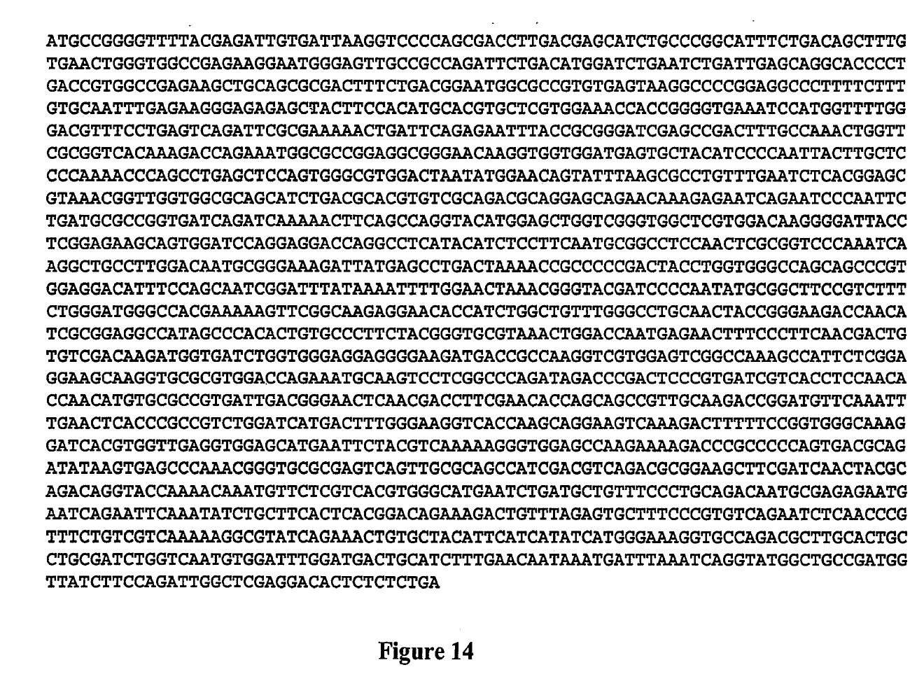

FIG. 15 shows the optimized nucleotide sequence encoding for the expression of AAV2 Rep78 protein in insect cells (SEQ ID NO. 4).

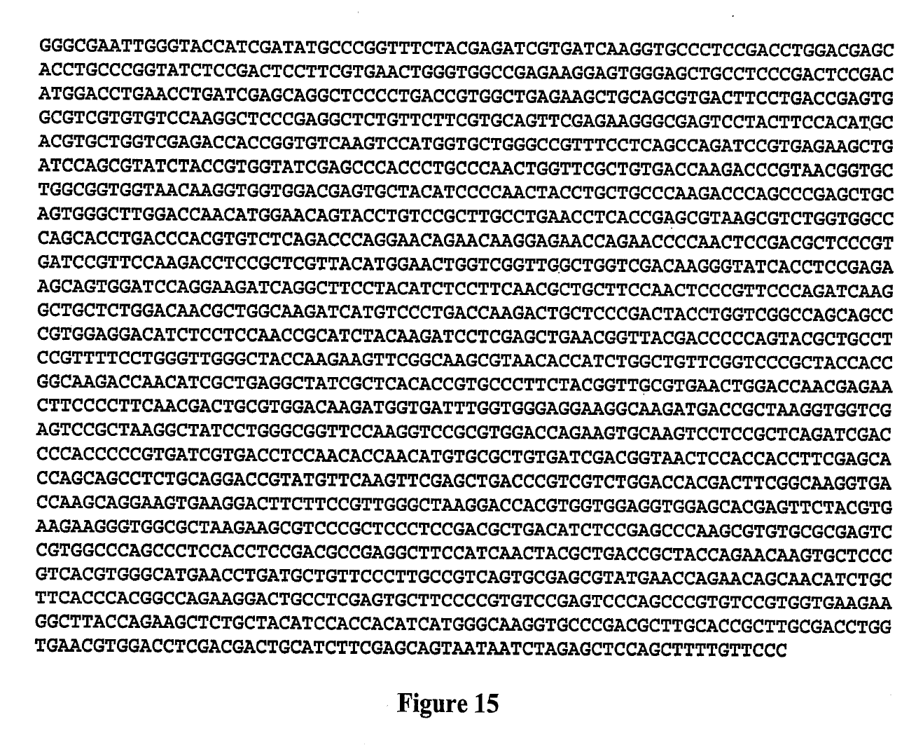

FIG. 16 shows the nucleotide sequence encoding for AAV capsid 2.5 (SEQ ID NO. 5).

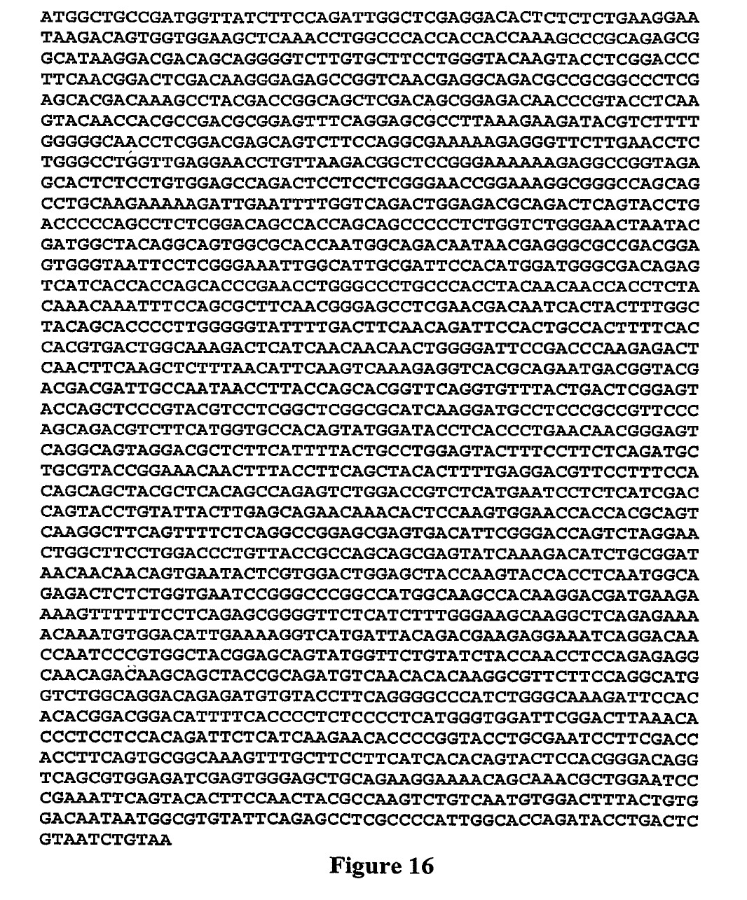

FIG. 17 shows the optimized nucleotide sequence encoding for the expression of the AAV capsid 2.5 in insect cells (SEQ ID NO. 6).

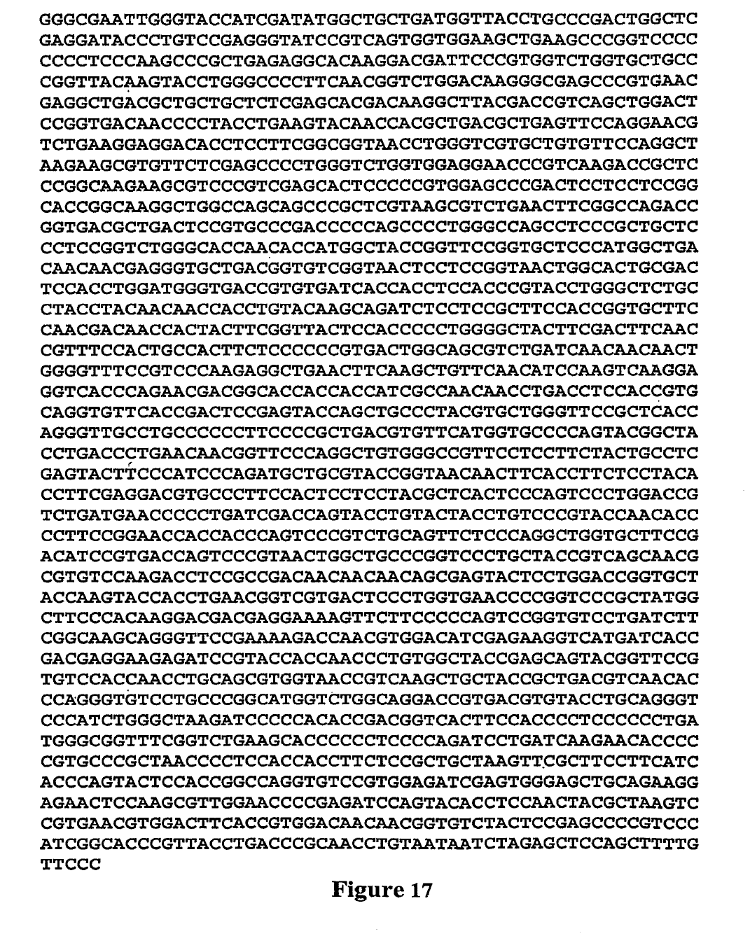

FIG. 18 shows the nucleotide sequence encoding for AAV8 VP1 protein (SEQ ID NO. 7).

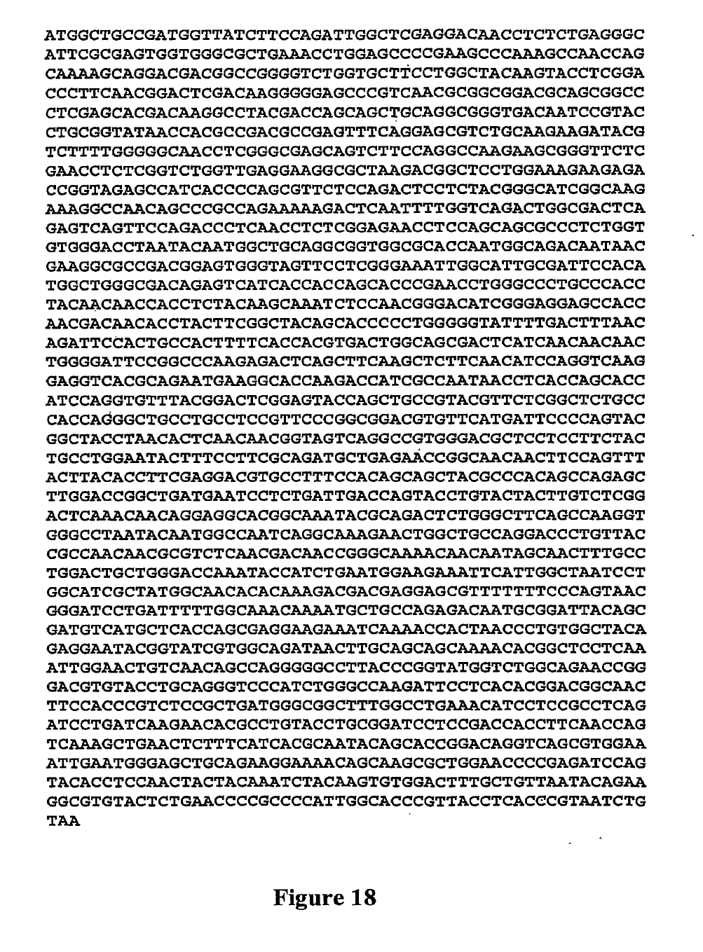

FIG. 19 shows the optimized nucleotide sequence encoding for the expression of the AAV8 VP1 protein in insect cells (SEQ ID NO. 8).

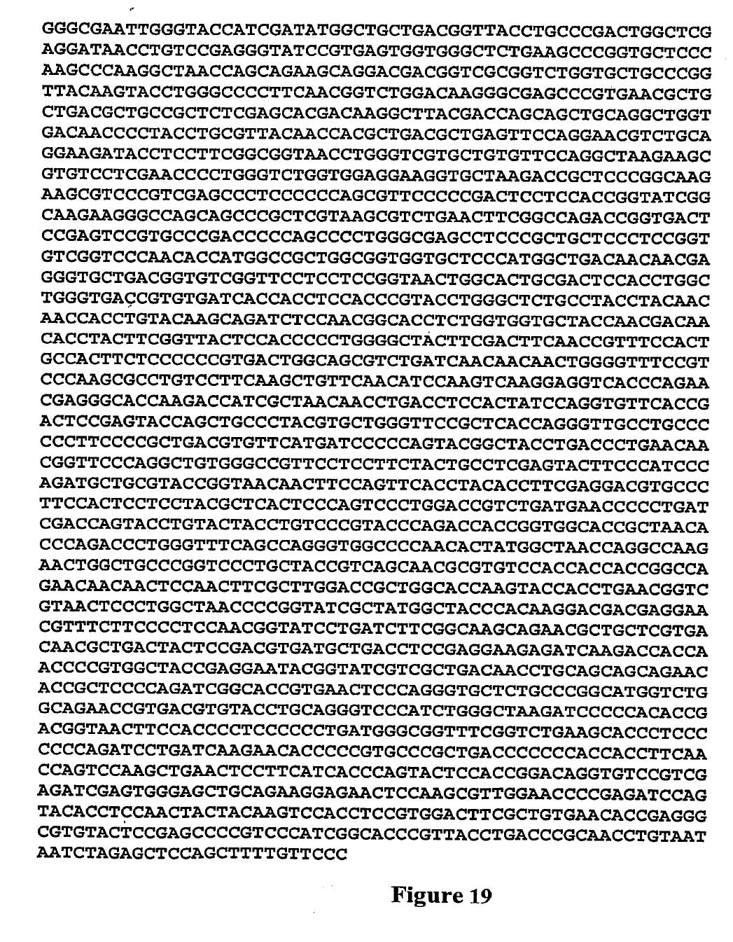

FIG. 20 shows the nucleotide sequence encoding for AAV9 VP1 protein (SEQ ID NO. 9).

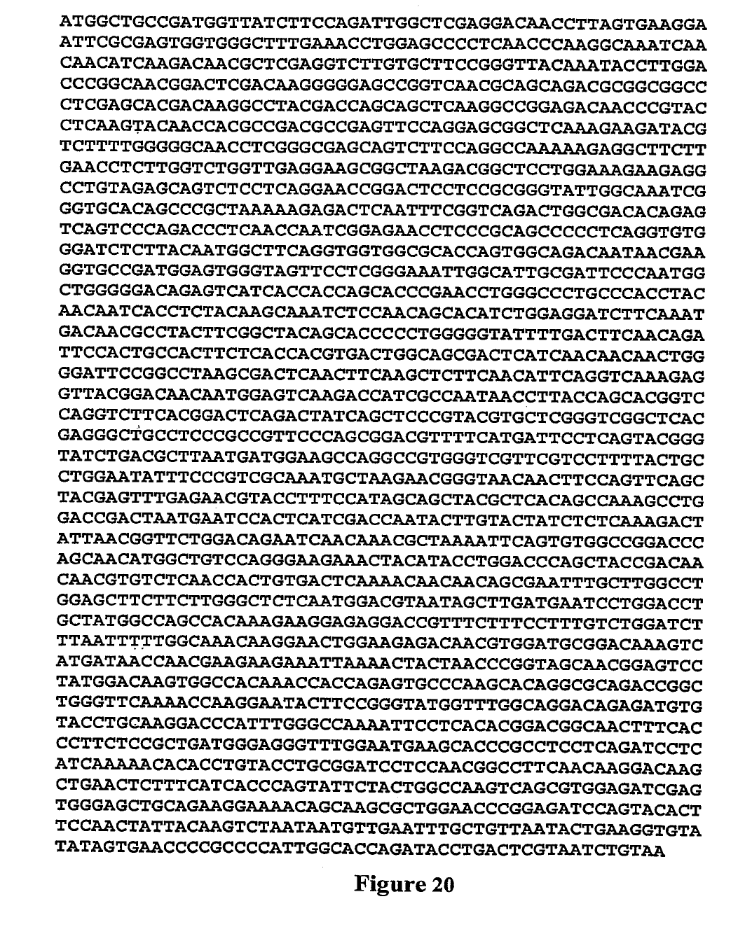

FIG. 21 shows the optimized nucleotide sequence encoding for the expression of AAV9 VP1 protein in insect cells (SEQ ID NO. 10).

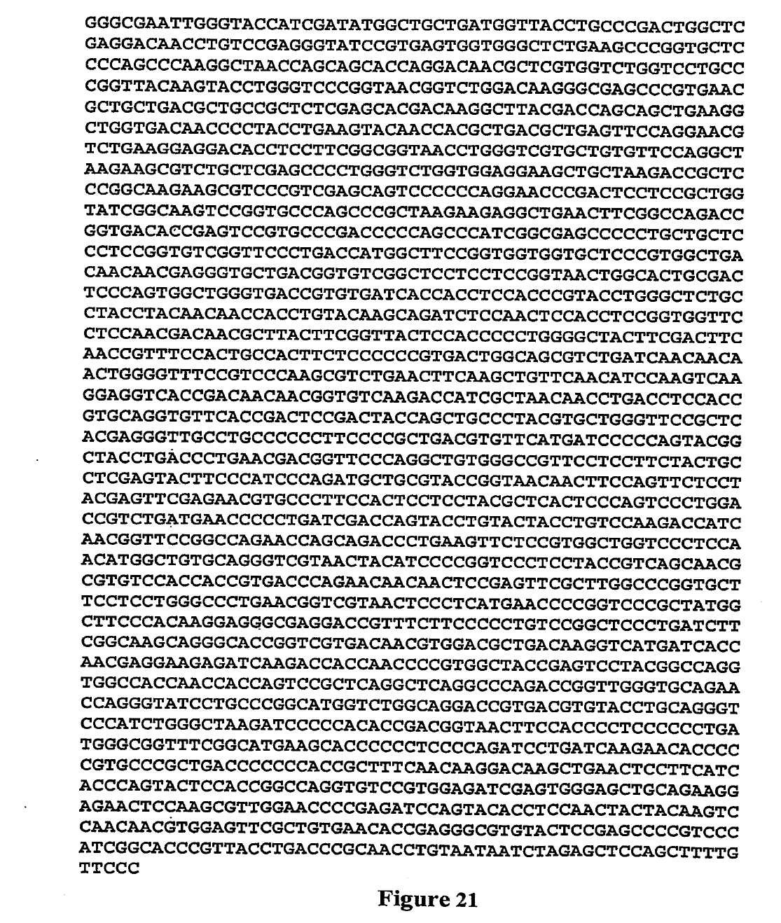

FIG. 22 shows the nucleotide sequence encoding for AAV2 VP1 Cap protein (SEQ ID NO. 11).

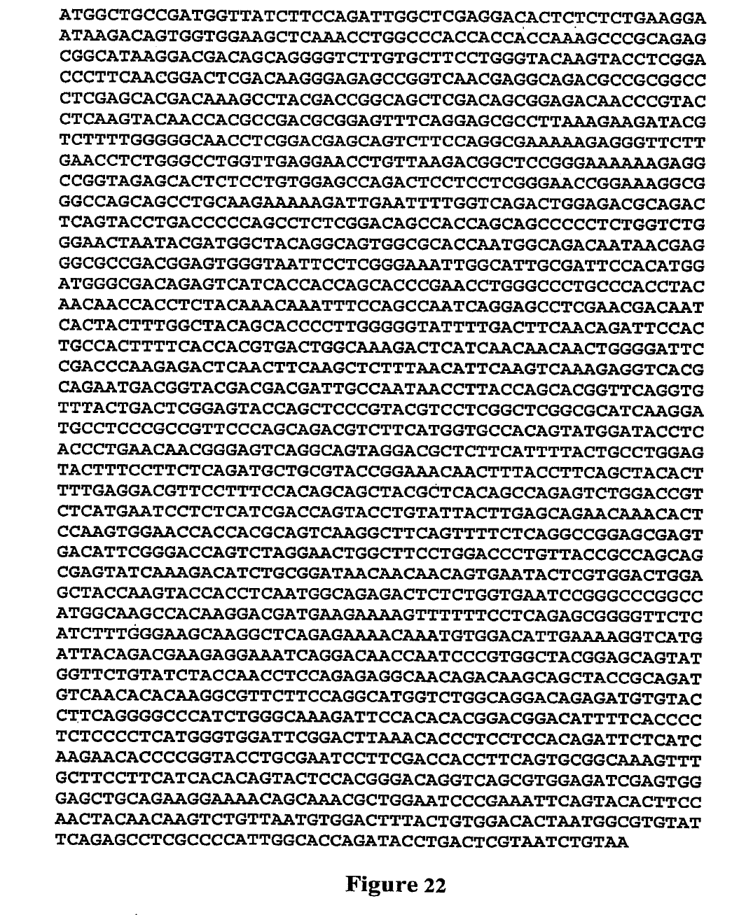

FIG. 23 shows the amino acid sequence for AAV2 VP1 Cap protein (SEQ ID NO. 12).

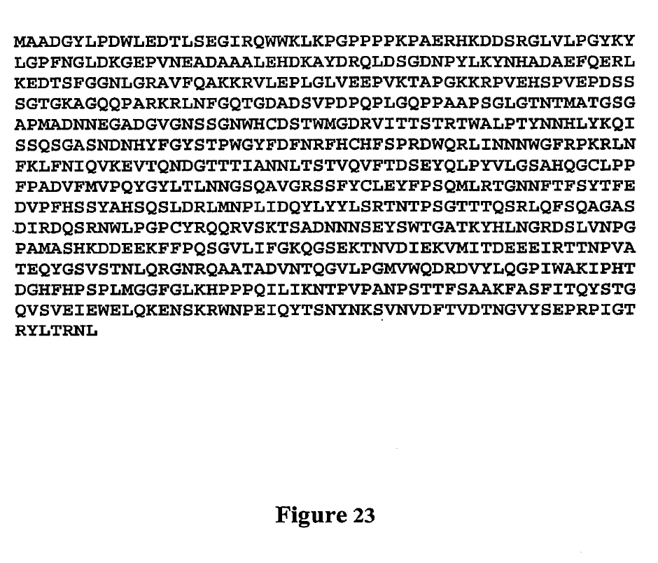

FIG. 24 shows the amino acid sequence for the AAV2.5 VP1 Cap protein in insect cells (SEQ ID NO. 13).

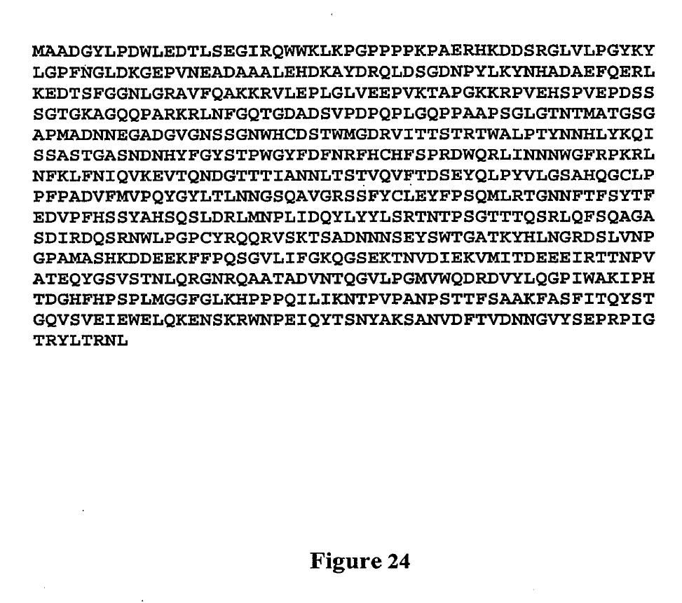

### DETAILED DESCRIPTION OF THE INVENTION

### Definitions

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. All publications, patent applications, patents, and other references mentioned herein are incorporated by reference in their entirety.

“AAV Cap” means AAV Cap proteins, VP1, VP2 and VP3 and analogs thereof.

“AAV Rep” means AAV Rep proteins and analogs thereof.

“AAV TR” means a palindromic sequence, comprising mostly complementary, symmetrically arranged sequences, and includes analogs of native AAV TRs and analogs thereof.

“Biologically-effective” with respect to an amount of a viral vector is an amount that is sufficient to result in infection (or transduction) and expression of the transgene in a target cell.

“Chimeric” means, with respect to a viral capsid or particle, that the capsid or particle includes sequences from different parvoviruses, preferably different AAV serotypes, as described in Rabinowitz et al., U.S. Pat. No. 6,491,907, entitled “Recombinant parvovirus vectors and method of making,” granted on Dec. 10, 2002, the disclosure of which is incorporated in its entirety herein by reference.

“Dependovirus” means the well-known genus containing the adeno-associated viruses (AAV), including but not limited to, AAV type 1, AAV type 2, AAV type 3, AAV type 4, AAV type 5, AAV type 6, avian AAV, bovine AAV, canine AAV, equine AAV, and ovine AAV. See, e.g., Bernard N. Fields et al., Virology, volume 2, chapter 69 (3d ed., Lippincott-Raven Publishers), the entire disclosure of which is incorporated herein by reference.

“Duplexed vectors” may interchangeably be referred to herein as “dimeric” or “self-complementary.” vectors. The duplexed parvovirus particles may, for example, comprise a parvovirus capsid containing a virion DNA (vDNA). The vDNA is self-complementary so that it may form a hairpin structure upon release from the viral capsid. The duplexed vDNA appears to provide to the host cell a double-stranded DNA that may be expressed (i.e., transcribed and, optionally, translated) by the host cell without the need for second-strand synthesis, as required with conventional parvovirus vectors. Examples of duplexed vectors suitable for use in the invention are described in U.S. Patent Publication No. 2004/0029106, entitled “Duplexed parvovirus vectors,” published on Feb. 12, 2004 in the name of Samulski et al., the entire disclosure of which is incorporated herein by reference. The duplexed vector genome preferably contains sufficient packaging sequences for encapsidation within the selected parvovirus capsid (e.g, AAV capsid). Those skilled in the art will appreciate that the duplexed vDNA may not exist in a double-stranded for under all conditions, but has the ability to do so under conditions that favor annealing of complementary nucleotide bases. “Duplexed parvovirus particle” encompasses hybrid, chimeric and targeted virus particles. Preferably, the duplexed parvovirus particle has an AAV capsid, which may further be a chimeric or targeted capsid, as described above.

“Expression control sequence” means one or more a nucleic acid sequences that regulates the expression of a nucleotide sequence to which it is operably linked. An expression control sequence is “operably linked” to a nucleotide sequence when the expression control sequence controls and/or regulates the transcription and/or the translation of the nucleotide sequence. Components of an expression control sequence can include, for example, promoter(s), enhancer(s), internal ribosome entry site(s) (IRES), transcription terminator(s), start codon(s), splicing signal for intron(s), and stop codon(s). The term “expression control sequence” is intended to include, at a minimum, a sequence designed to influence expression, and can also include other component related to transcription, translation, translocation, secretion, isolation and the like, such as leader sequences and fusion partner sequences. Expression control sequences are preferably designed to minimize or eliminate undesirable potential initiation codons in and out of frame as well as undesirable potential splice sites. Sequences, such as polyadenylation sequences (pA), can be included to provide for the addition of a polyA tail, i.e., a string of adenine residues at the 3′-end of an mRNA, sequences referred to as polyA sequences. The expression control sequences can be designed to enhance mRNA stability. Expression control sequences which affect transcription and translation stability, e.g., promoters, as well as sequences which effect the translation, e.g., Kozak sequences, are known in insect cells. The expression control sequence(s) can be designed to modulate expression of the nucleotide sequence to which they are operably linked by increasing or decreasing expression levels as needed.

“Flanked,” with respect to a sequence that is flanked by other elements, indicates the presence of one or more the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence (e.g., a transgene) that is “flanked” by two other elements (e.g., TRs), indicates that one element is located 5′ to the sequence and the other is located 3′ to the sequence; however, there may be intervening sequences therebetween.

“Hybrid” means, with respect to a viral particle, a viral particle in which the viral TRs and viral capsid are from different parvoviruses. Preferably, the viral TRs and capsid are from different serotypes of AAV (e.g., as described in international patent publication WO 00/28004, and Chao et al., (2000) Molecular Therapy 2:619; the disclosures of which are incorporated herein in their entireties).

“Insect cell-compatible,” with respect to a viral vector, helper functions or packaging functions, means any nucleic acid sequence which facilitates transformation or transfection of an insect cell with a nucleic acid and/or expression of a heterologous nucleic acid within such insect cell.

“Packaged viral vector” and the like refers to a virus particle, such as a parvovirus particle, that functions as a delivery vehicle for a nucleic acid sequence, such as a recombinant viral vector comprising a transgene and associated expression control sequence(s) flanked by TRs, which is packaged within a virus capsid.

“Parvovirus” means family Parvoviridae, including without limitation autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, mouse minute virus, bovine parvovirus, canine parvovirus, chicken parvovirus, feline panleukopenia virus, feline parvovirus, goose parvovirus, and B19 virus. Other autonomous parvoviruses are known to those skilled in the art. See, e.g., Bernard N. Fields et al., Virology, volume 2, chapter 69 (3d ed., Lippincott-Raven Publishers); S. J. Flint, et al., Principles of Virology (2nd ed., ASM Press, 2004) for their teaching on the characterization of the Parvoviridae family. The genomic sequences (and corresponding amino acid sequences) of the various autonomous parvoviruses and the different serotypes of AAV, as well as the sequences of the TRs, and the Cap and Rep polypeptides are known in the art. Such sequences may be found in the literature or in public databases such as GenBank. See, e.g., GenBank Accession Numbers NC 002077, NC 001863, NC 001862, NC 001829, NC 001729, NC 001701, NC 001510, NC 001401, AF063497, U89790, AF043303, AF028705, AF028704, J02275, J01901, J02275, X01457, AF288061, AH009962, AY028226, AY028223, NC 001358, NC 001540, the disclosures of which are incorporated herein in their entirety. See also, e.g., Chiorini et al., (1999) J. Virology 73:1309; Xiao et al., (1999) J. Virology 73:3994; Muramatsu et al., (1996) Virology 221:208; international patent publications WO 00/28061, WO 99/61601, WO 98/11244; and U.S. Pat. No. 6,156,303, the disclosures of which are incorporated herein in their entirety. An early description of the AAV1, AAV2 and AAV3 TR sequences is provided by Xiao, X., (1996), “Characterization of Adeno-associated virus (AAV) DNA replication and integration,” Ph.D. Dissertation, University of Pittsburgb, the disclosure of which is incorporated herein it its entirety. The viral vectors and viral capsids are described in more detail in the ensuing sections.

“Polypeptide” encompasses both peptides and proteins, unless indicated otherwise.

“Recombinant” means a genetic entity distinct from that generally found in nature. As applied to a polynucleotide or gene, this means that the polynucleotide is the product of various combinations of cloning, restriction and/or ligation steps, and other procedures that result in the production of a construct that is distinct from a polynucleotide found in nature.

“Recombinant viral vector” means a recombinant polynucleotide vector comprising one or rnore heterologous sequences (i.e., polynucleotide sequence not of viral origin). In the case of recombinant parvovirus vectors, the recombinant polynucleotide is flanked by at least one, preferably two, inverted terminal repeat sequences (ITRs).

“Substantial homology” or “substantial similarity,” means, when referring to a nucleic acid or fragment thereof, indicates that, when optimally aligned with appropriate nucleotide insertions or deletions with another nucleic acid (or its complementary strand), there is nucleotide sequence identity in at least about 95 to 99% of the sequence.

“Targeted” means, with respect to a viral capsid or particle, a capsid or particle having a directed tropism, e.g., as described in International Patent Publication No. WO 00/28004, the entire disclosure of which is incorporated herein by reference.

“Therapeutic polypeptide” or “therapeutic product” means a polypeptide that may alleviate, reduce or delay the onset of symptoms that result from an absence or defect in a polypeptide in a cell or subject. Alternatively, a “therapeutic polypeptide” is one that otherwise confers a benefit to a subject, e.g., anti-cancer effects or improvement in transplant survivability.

“Transduction” or “infection” of a cell by a virus means that the virus enters the cell to establish a latent or active (i.e., lytic) infection.

“Transfection” of a cell means that genetic material is introduced into a cell for the purpose of genetically modifying the cell. Transfection can be accomplished by a variety of means known in the art, such as transduction or electroporation.

“Transgene” is used in a broad sense to mean any heterologous nucleotide sequence incorporated in a viral vector for expression in a target cell and associated expression control sequences, such as promoters. It is appreciated by those of skill in the art that expression control sequences will be selected based on ability to promote expression of the transgene in the target cell. An example of a transgene is a nucleic acid encoding a therapeutic polypeptide.

“Vector,” means a recombinant plasmid or virus that comprises a polynucleotide to be delivered into a host cell, either in vitro or in vivo.

The invention described here generally relates to the cells, genetic constructs, processes and strategies for the production of packaged parvovirus vectors. Parvovirus vectors are highly useful as research tools for the study of gene and polypeptide expression, and also show great promise in the realm of gene therapy. However, working with parvovirus vectors is technically challenging and time-consuming, rendering use of them less efficient and cost-effective than is desirable. One particular hindrance to the use of parvovirus vectors is the time- and labor-intensive process required for production of infective parvovirus particles. The production strategies of the invention generally relate to culturing packaging cells of the invention to produce packaged viral vectors. The packaging cells of the invention generally include cells with the following packaging cell functions: (1) viral vector function(s), (2) packaging function(s), and (3) helper function(s). The methods of AAV production generally involve (1) providing the component functions, (2) introducing the component functions into a compatible cell, and (3) maintaining the cell under conditions sufficient to produce the AAV. Each of the packaging cell functions is discussed in the ensuing sections. The methods of the invention are useful in the production of packaged viral vectors. In general, packaged viral vectors include a viral vector packaged in a capsid, such as a parvovirus capsid, a targeted AAV capsid, or a chimeric AAV capsid. Viral vectors and viral capsids and the components of a packaged viral vector are discussed more fully in the ensuing sections.

Packaging Cells

As noted above, the packaging cells of the invention generally include cells with the following packaging cell functions: (1) viral vector function(s), (2) packaging function(s), (3) helper function(s), and (4) associated expression control sequences. During production, the packaging cells generally include one or more viral vector functions along with packaging functions and helper functions sufficient to result in expression and packaging of the viral vector. These various functions may be supplied together or separately to the packaging cell using a genetic construct such as a plasmid or an amplicon, and they may exist extrachromosomally within the cell line or integrated into the cell's chromosomes. The cell lines may be supplied with any one or more of the stated functions already incorporated, e.g., a cell line with one or more vector functions incorporated extrachromosomally or integrated into the cell's chromosomal DNA, a cell line with one or more packaging functions incorporated extrachromosomally or integrated into the cell's chromosomal DNA, or a cell line with helper functions incorporated extrachromosomally or integrated into the cell's chromosomal DNA. Nucleotide sequences encoding the packaging cell functions, such as transposition proteins, are operably linked to at least one expression control sequence for expression in an insect cell.

Any method of introducing one or more nucleotide sequences carrying the packaging cell functions into a cellular host for replication and packaging may be employed, including but not limited to, electroporation, calcium phosphate precipitation, microinjection, cationic or anionic liposomes, and liposomes in combination with a nuclear localization signal. In embodiments wherein the packaging functions are provided by transfection using a virus vector; standard methods for producing viral infection may be used.

The nucleotide sequences of the invention can be stably introduced into an insect genome. Incorporation of the nucleotide sequences of the invention into the genome may be aided by, for example, the use of a vector comprising nucleotide sequences highly homologous to regions of the insect genome. The use of specific sequences, such as transposons, is another way to introduce a nucleotide sequence into a genome. Often, a cell which underwent such “transformation,” i.e., addition of a nucleic acid sequence to the cell, is selected or identified by expression of a marker gene which, usually, is encoded by the nucleic acid sequence added to the cell. The incorporation of the nucleic acid sequence in the genome then can be determined by, for example, Southern blots or polymerase chain reaction (PCR) methods.

Viral Vector Functions

The viral vector component of the packaged viral vectors of the invention typically includes at least one transgene and associated expression control sequences for controlling expression of the transgene. The viral vector may include cis-acting functions sufficient to enable integration of the transgene into the genome of a target cell. Typically, the viral vector includes a portion of a parvovirus genome, such as an AAV genome with rep and cap deleted and replaced by the transgene and its associated expression control sequences. The transgene is typically flanked by two AAV TRs, in place of the deleted viral rep and cap ORFs. Appropriate expression control sequences are included, such as a tissue-specific promoter and other regulatory sequences suitable for use in facilitating tissue-specific expression of the transgene in the target cell. The transgene is typically a nucleic acid sequence that can be expressed to produce a therapeutic polypeptide or a marker polypeptide. The viral vector may be any suitable nucleic acid construct, such as a DNA or RNA construct and may be single stranded, double stranded, or duplexed.

The viral vector functions may suitably be provided as duplexed vector templates, as described in U.S. Patent Publication No. 2004/0029106 to Samulski et al. (the entire disclosure of which is incorporated herein by reference for its teaching regarding duplexed vectors). Duplexed vectors are dimeric self-complementary (sc) polynucleotides (typically, DNA). For example, the DNA of the duplexed vectors can be selected so as to form a double-stranded hairpin structure due to intrastrand base pairing. Both strands of the duplexed DNA vectors may be packaged within a viral capsid. The duplexed vector provides a function comparable to double-stranded DNA virus vectors and can alleviate the need of the target cell to synthesize complementary DNA to the single-stranded genome normally encapsidated by the virus.

The TR(s) (resolvable and non-resolvable) selected for use in the viral vectors are preferably AAV sequences, with serotypes 1, 2, 3, 4, 5 and 6 being preferred. Resolvable AAV TRs need not have a wild-type TR sequence (e.g., a wild-type sequence may be altered by insertion, deletion, truncation or missense mutations), as long as the TR mediates the desired functions, e.g., virus packaging, integration, and/or provirus rescue, and the like. The TRs may be synthetic sequences that function as AAV inverted terminal repeats, such as the “double-D sequence” as described in U.S. Pat. No. 5,478,745 to Samulski et al., the entire disclosure of which is incorporated in its entirety herein by reference. Typically, but not necessarily, the TRs are from the same parvovirus, e.g., both TR sequences are from AAV2.

Packaging Functions

The packaging functions include genes, such as AAV rep and cap, for viral vector replication and packaging. Packaging functions may, for example, include functions necessary or useful for viral gene expression, viral vector replication, rescue of the viral vector from the integrated state, viral gene expression, and packaging of the viral vector into a viral particle. The packaging functions may be supplied together or separately to the packaging cell using a genetic construct, such as a plasmid or an amplicon. The packaging functions may exist extrachromosomally within the packaging cell, but are preferably integrated into the cell's chromosomal DNA.

Baculovirus packaging functions may include functions required to generate recombinant baculoviruses, such as, found in the Bac-Bac® expression system (Invitrogen) and described by Luckow, et al. 1993, J. Virol. 67, 4566, including a control expression plasmid containing the Gus and/or CAT gene which express either β-glucuronidase and/or chloramphenicol acetyl-transferase for production of a recombinant baculovirus.

Sequences from more than one AAV serotype can be combined for production of AAV. For example, the AAV TR nucleotide sequence can be derived from one serotype, for example AAV2, while any of the other nucleotide sequences can comprise open reading frames or coding sequences derived from one or more other serotypes, for example, serotype 3. AAV serotypes 1, 2, 3, 4 and 5 are examples of suitable sources of AAV nucleotide sequences for use in the context of the present invention.

Capsid Components

The packaging functions include capsid components. The capsid components are preferably from a parvoviral capsid, such as an AAV capsid or a chimeric AAV capsid function. Examples of suitable parvovirus viral capsid components are capsid components from the family Parvoviridae, such as an autonomous parvovirus or a Dependovirus. For example, the capsid components may be selected from AAV capsids, e.g., AAV1, AAV2, AAV3, AAV4, AAV5 and/or AAV6 capsids. Capsid components may include components from two or more AAV capsids.

Further, the inventors have surprisingly discovered that that supplementation of VP1 relative to VP2 and VP3 results in the production of a greater percentage of infectious particles. In one embodiment, the cells are supplemented with moi of VP1 that provides an about 1, 2 or 3 extra VP1 vectors to at least 100% of cells in the culture. Regardless of how the supplementation of VP1 is accomplished, the method produces at least about 10, 20, 30, 40, 50, 60, 70, 80, 90, 100, 110, 120, 130, 140, 15, 160, 170, 180, 190 or 200 times more infectious viral particles than in the absence of the supplementation.

It will be appreciated that this VP1 supplementation can be achieved in a variety of ways. For example, the method of the invention may include (a) providing an insect cell; (b) introducing into the insect cell one or more vectors comprising nucleotide sequences encoding: (i) a transgene flanked by TRs; and (ii) baculovirus packaging functions comprising Rep components and Cap components sufficient to result in packaging of infective parvovirus particles, wherein VP1 is supplemented relative to VP2 and VP3 sufficient to increase the production of infectious viral particles; (c) introducing into the cell a nucleic acid encoding baculovirus helper functions for expression in the insect cell; and (d) culturing the cell under conditions sufficient to produce the infectious packaged parvovirus vector. In one approach, illustrated by the examples below, a Cap vector provides VP1/VP2/VP3 and a second VP1 vector supplements VP1 produced by the Cap vector. Thus, in this approach, the supplementation can be effected by (a) introducing into the insect cell a Cap vector comprising one or more nucleotide sequences expressing VP1, VP2 and VP3; and (b) introducing into the insect cell a VP1 vector comprising nucleotide sequences expressing VP1. The Cap vector and the VP1 vector can typically be introduced into the insect cell at a moi which is at least 1.

The inventors have found that overexpression of VP1 can result in a high level of particle degradation. Therefore, in one embodiment of the invention, expression control sequences of the VP1 vector provide relatively weaker expression as compared to expression control sequences of the Cap vector. For example, expression of the VP1 vector is suitably from about 1 to about 75% of the expression of the Cap vector, from about 1 to about 50% of the expression of the Cap vector, or from about 1 to about 25% of the expression of the Cap vector. Weaker expression may be provided, for example, using a mutated polyhedrin promoter which provides. Ideally, the supplementation of VP1 results in production of a molecular ratio of approximately 10:10:80 VP1:VP2:VP3. The supplementation of VP1 may result in production of infectious packaged parvovirus vectors in an amount which is about 10, 20, 30, 40, 50, 60, 70, 80, 90, 100, 110, 120, 130, 140, 15, 160, 170, 180, 190 or 200 times greater than in corresponding method in the absence of the supplementation.

The choice of capsid components is generally based on considerations such as the target cell type, the desired level of expression, the nature of the heterologous nucleotide sequence to be expressed, issues related to viral production, and the like. For example, the AAV1 capsid may be advantageously employed for targeting of skeletal muscle, liver and cells of the central nervous system (e.g., brain); AAV5 for targeting cells of the airway and lung; AAV3 for targeting bone marrow cells; and AAV4 for particular cells of the brain (e.g., appendable cells). A full complement of AAV VP capsid proteins comprises VP1, VP2, and VP3. The ORF comprising nucleotide sequences encoding AAV VP capsid proteins may comprise less than a full complement of VP proteins. However, in a preferred embodiment, the full complement of VP proteins is provided. The VP capsid proteins may be provided in different ORFs on the same vector and/or on different vectors.

In a more preferred embodiment, one or more of the VP capsid proteins is a chimeric protein, comprising amino acid sequences from two or more viruses, preferably two or more AAVs, as described in Rabinowitz et al., U.S. Pat. No. 6,491,907, entitled “Recombinant parvovirus vectors and method of making,” granted on Dec. 10, 2002, the entire disclosure of which is incorporated in its entirety herein by reference.

For example, the chimeric virus capsid can include a capsid region from an adeno-associated virus (AAV) and at least one capsid region from a B19 virus. The chimeric capsid can, for example, include an AAV capsid with one or more B19 capsid subunits, e.g., an AAV capsid subunit can be replaced by a B19 capsid subunit. For example, in a preferred embodiment, the VP1, VP2 or VP3 subunit of the AAV capsid can be replaced by the VP1, VP2 or VP3 subunit of B19. As another example, the chimeric capsid may include an AAV type 2 capsid in which the type 2 VP1 subunit has been replaced by the VP1 subunit from an AAV type 1, 3, 4, 5, or 6 capsid, preferably a type 3, 4, or 5 capsid. Alternatively, the chimeric parvovirus has an AAV type 2 capsid in which the type 2 VP2 subunit has been replaced by the VP2 subunit from an AAV type 1, 3, 4, 5, or 6 capsid, preferably a type 3, 4, or 5 capsid. Likewise, chimeric parvoviruses in which the VP3 subunit from an AAV type 1, 3, 4, 5 or 6 (more preferably, type 3, 4 or 5) is substituted for the VP3 subunit of an AAV type 2 capsid are preferred. As a further alternative, chimeric parvoviruses in which two of the AAV type 2 subunits are replaced by the subunits from an AAV of a different serotype (e.g., AAV type 1, 3, 4, 5 or 6) are preferred. In exemplary chimeric parvoviruses according to this embodiment, the VP1 and VP2, or VP1 and VP3, or VP2 and VP3 subunits of an AAV type 2 capsid are replaced by the corresponding subunits of an AAV of a different serotype (e.g., AAV type 1, 3, 4, 5 or 6). Likewise, in other preferred embodiments, the chimeric parvovirus has an AAV type 1, 3, 4, 5 or 6 capsid (preferably the type 2, 3 or 5 capsid) in which one or two subunits have been replaced with those from an AAV of a different serotype, as described above for AAV type 2.

In still other embodiments, the minor subunit of one parvovirus may be substituted with any minor subunit of another parvovirus (e.g., VP2 of AAV type 2 may be replaced with VP1 from AAV type 3; VP1 of B19 may substitute for VP1 and/or VP2 of AAV). Likewise, the major capsid subunit of one parvovirus may be replaced with the major capsid subunit of another parvovirus. The present invention further provides chimeric parvoviruses comprising an AAV capsid in which a loop region(s) in the major VP3 subunit is replaced by a loop region (s) (preferably, a corresponding loop region(s)) from the major subunit of an autonomous parvovirus. In particular, the loop region 1, 2, 3 and/or 4 from an AAV type 1, 2, 3, 4, 5, or 6 VP3 subunit is replaced with a loop region from the major subunit of an autonomous parvovirus.

A particularly preferred chimeric viral capsid includes the AAV2.5 capsid, which includes the nucleotide sequence encoding for the AAV2.5 VP1 capsid protein, wherein the expressed protein has the following mutations: 263 Q→A; 265 insertion T; 705 N→A; 708 V→A; and 716 T→N (SEQ ID NO. 13).

Replication Components

The packaging functions also include replication components. For example, the replication components may include Rep78, Rep68, Rep52, Rep40 and/or various analogs thereof. It is possible to use less than the four Rep enzymes, such as only one of the Rep78/Rep68 enzymes and only one of the Rep52/Rep40 enzymes. Preferably, the Rep sequences expressed in the insect cell are Rep78 and Rep52.

Helper Functions

The packaging cell functions also include helper functions. The helper functions include helper virus elements needed for establishing active infection of the packaging cell. The presence of helper functions is required to initiate packaging of the viral vector. Examples include functions derived from adenovirus, baculovirus and/or herpes virus sufficient to result in packaging of the viral vector. For example, adenovirus helper functions will typically include adenovirus components E1a, E1b, E2a, E4, and VA RNA. The packaging functions may be supplied by infection of the packaging cell with the required virus. The packaging functions may be supplied together or separately to the packaging cell using a genetic construct such as a plasmid or an amplicon. The packaging functions may exist extrachromosomally within the packaging cell, but are preferably integrated into the cell's chromosomal DNA.

The multiplicity of infection (MOI) and the duration of the infection will depend on the type of virus used and the packaging cell line employed. Any suitable helper vector may be employed. The vector can be introduced into the packaging cell by any suitable method known in the art. In a preferred method in which insect cells serve as the packaging cell, baculovirus may serve as a helper virus.

A suitable method for providing helper functions employs a non-infectious adenovirus miniplasmid that carries all of the helper genes required for efficient AAV production (Ferrari et al., (1997) Nature Med. 3:1295; Xiao et al., (1998) J. Virology 72:2224). The rAAV titers obtained with adenovirus miniplasmids are forty-fold higher than those obtained with conventional methods of wild-type adenovirus infection (Xiao et al., (1998) J. Virology 72:2224). This approach obviates the need to perform co-transfections with adenovirus (Holscher et al., (1994), J. Virology 68:7169; Clark et al., (1995) Hum. Gene Ther. 6:1329; Trempe and Yang, (1993), in, Fifth Parvovirus Workshop, Crystal River, Fla.).

Herpes virus may also be used as a helper virus in AAV packaging methods. Hybrid herpes viruses encoding the AAV Rep protein(s) may advantageously facilitate for more scalable AAV vector production schemes. A hybrid herpes simplex virus type I (HSV-1) vector expressing the AAV-2 rep and cap genes has been described (Conway et al., (1999) Gene Therapy 6:986 and WO 00/17377, the disclosures of which are incorporated herein in their entireties).

Any method of introducing the nucleotide sequence carrying the helper functions into a cellular host for replication and packaging may be employed, including but not limited to, electroporation, calcium phosphate precipitation, microinjection, cationic or anionic liposomes, and liposomes in combination with a nuclear localization signal. In embodiments wherein the helper functions are provided by transfection using a virus vector or infection using a helper virus; standard methods for producing viral infection may be used.

Expression Control Sequences

The viral vector function(s), packaging function(s), and helper function(s), are each operably linked to one or more associated expression control sequences, such as one or more promoter sequences, translation initiation sequences, and stop codons. For production in insect cells, transcriptional promoters compatible with insect cell gene expression can be employed. Expression control sequences are selected to maximize the production of infective viral particles.

Cell Lines

Preferred cell lines for use as packaging cells are insect cell lines, preferably cells from the order Lepidoptera or cells derived from cells of this order. Any insect cell which allows for replication of AAV and which can be maintained in culture can be used in accordance with the present invention. Preferably, the insect cells are from the genus Spodoptera or Trichopulsia. Examples include Spodoptera frugiperda, such as the Sf9 or Sf21 cell lines, Drosophila spp. cell lines, or mosquito cell lines, e.g., Aedes albopictus derived cell lines. A preferred cell line is the Spodoptera frugiperda Sf9 cell line. Other examples include High Five™ cells (BRI-TN-5B1-4), and Mimic-SF9 cells. Cells derived from any of the cells listed herein may also be useful in the practice of the invention.

The following references are incorporated herein for their teachings concerning use of insect cells for expression of heterologous polypeptides, methods of introducing nucleic acids into such cells, and methods of maintaining such cells in culture: Methods in Molecular Biology, ed. Richard, Humana Press, NJ (1995); O'Reilly et al., Baculovirus Expression Vectors: A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059.

Growing Cells

Growing conditions for insect cells in culture, and production of heterologous products in insect cells in culture are described in Richard (1995), supra; O'Reilly et al., (1994) supra; Sarnulski et al., (1989) supra; Kajigaya et al., (1991) supra; Ruffing et al., (1992) supra; Kirnbauer et al., (1996) supra; Zhao et al., (2000) supra; and Samulski et al., U.S. Pat. No. 6,204,059.

Production Strategies

The invention provides methods of making packaged viral vectors. The methods generally include introducing into an insect cell the following packaging cell functions: (1) viral vector function(s), (2) packaging function(s), (3) helper function(s), and (4) associated expression control sequences. The functions should be sufficient to result in production of encapsidated viral vector. A variety of configurations for providing these functions are possible within the scope of the present invention. In each case, the resulting packaging cells are then incubated to produce the packaged viral vectors, and the packaged viral vectors may then be isolated using isolation techniques known in the art.

Any method of introducing the nucleotide sequence carrying the viral vector functions, packaging functions and helper functions into a cellular host for replication and packaging may be employed, including but not limited to, electroporation, calcium phosphate precipitation, microinjection, cationic or anionic liposomes, and liposomes in combination with a nuclear localization signal. In embodiments wherein the viral vector functions are provided by transfection using a virus vector; standard methods for producing viral infection may be used.

The resulting packaging cells are themselves an aspect of the invention. The invention also includes a method of manufacturing infective AAV particles, wherein the packaging cells are maintained under conditions sufficient to produce infective particles.

Purification of Packaged Viral Vectors

Vector stocks free of contaminating helper virus may be obtained by any method known in the art. For example, duplexed virus and helper virus may be readily differentiated based on size. The duplexed virus may also be separated away from helper virus based on affinity for a heparin substrate (Zolotukhin et al. (1999) Gene Therapy 6:973). Preferably, deleted replication-defective helper viruses are used so that any contaminating helper virus is not replication competent. As a further alternative, an adenovirus helper lacking late gene expression may be employed, as only adenovirus early gene expression is required to mediate packaging of the duplexed virus. Adenovirus mutants defective for late gene expression are known in the art (e.g., ts100K and ts149 adenovirus mutants).

Product of Interest

Generally, a product of interest is a gene product which can be a polypeptide, RNA molecule, or other gene product that is desired for expression in a mammalian cell or an insect cell. A product of interest can include, for example, polypeptides that serve as marker polypeptides to assess cell transformation and expression, fusion proteins, polypeptides having a desired biological activity, gene products that can complement a genetic defect, RNA molecules, transcription factors, and other gene products that are of interest in regulation and/or expression. For example, gene products of interest include nucleotide sequences that provide a desired effect or regulatory function (e.g., transposons, transcription factors). Examples of gene products of interest include, but are not limited to: hormone receptors (e.g., mineralcorticosteroid, glucocorticoid, and thyroid hormone receptors); intramembrane proteins (e.g., TM-1 and TM-7); intracellular receptors (e.g., orphans, retinoids, vitamin D3 and vitamin A receptors); signaling molecules (e.g., kinases, transcription factors, or molecules such signal transducers and activators of transcription receptors of the cytokine superfamily (e.g. erythropoietin, growth hormone, interferons, and interleukins, and colony-stimulating factors; G-protein coupled receptors, e.g., hormones, calcitonin, epinephrine, gastrin, and paracrine or autocrine mediators, such as stomatostatin or prostaglandins; neurotransmitter receptors (norepinephrine, dopamine, serotonin or acetylcholine); pathogenic antigens, which can be of viral, bacterial, allergenic, or cancerous origin; and tyrosine kinase receptors (such as insulin growth factor, and nerve growth factor). Gene products currently used in AAV-mediated gene therapy trials also are important gene products (e.g., CFTR and Factor IX).

A gene product of interest can be a therapeutic gene product. A therapeutic gene product is a polypeptide, RNA molecule, or other gene product that, when expressed in a target cell, provides a desired therapeutic effect, e.g., ablation of an infected cell, expression of a polypeptide having a desired biological activity, and/or expression of an RNA molecule for antisense therapy (e.g., regulation of expression of a endogenous or heterologous gene in the target cell genome). For example, Goldsmith et al., WO 90/07936, described a system for ablating specific cells within a tissue by using a promoter that is activated only in that tissue to express a therapeutic gene product only in the desired cells. For example, in a patient about to receive a heterologous transplant or graft, one may administer a polynucleotide encoding a toxin to T cells targeting the graft.

An AAV protein can be a gene product of interest. For example, the sequence of a Rep protein, such as Rep78 or Rep68, or a functional fragment thereof can be a gene product of interest for expression in a mammalian cell or an insect cell. A nucleic acid sequence encoding Rep78 and/or Rep68, if present in the viral vector and expressed in a mammalian cell or insect cell transduced with the rAAV produced in accordance with the present invention, allows for integration of the rAAV into the genome of the transduced mammalian cell or insect cell. Expression of Rep78 and/or Rep68 in an rAAV-transduced or infected mammalian cell or insect cell can bestow an advantage for certain uses of the rAAV, by allowing long term or permanent expression of any other gene product of interest introduced into the cell by the rAAV.

A selectable marker is one type of a gene product of interest. A selectable marker is a gene sequence or a polypeptide encoded by that gene sequence. Expression of the polypeptide encoded by the selectable marker allows a host cell transfected with an expression vector which includes the selectable marker to be easily identified from a host cell which does not have an expression vector encoding the selectable marker. An example is a host cell which can use the selectable marker to survive a selection process that would otherwise kill the host cell, such as treatment with an antibiotic. Such a selectable marker can be one or more antibiotic resistance factors, such as neomycin resistance (e.g., neo), hygromycin resistance, and puromycin resistance. A selectable marker also can be a cell-surface marker, such as nerve growth factor receptor or truncated versions thereof. Cells that express the cell-surface marker then can be selected using an antibody targeted to the cell-surface marker. The antibody targeted to the cell surface marker can be directly labeled (e.g., with a fluorescent substrate) or can be detected using a secondary labeled antibody or substrate which binds to the antibody targeted to the cell-surface marker. Alternatively, cells can be negatively selected by using an enzyme, such as Herpes simplex virus thymidine kinase (HSVTK) that converts a pro-toxin (gancyclovir) into a toxin or bacterial Cytosine Deaminase (CD) which converts the pro-toxin 5′-fluorocytosine (5′-FC) into the toxin 5′-fluorouracil (5′-FU). Alternatively, any nucleic acid sequence encoding a polypeptide can be used as a selectable marker as long as the polypeptide is easily recognized by an antibody.

The nucleic acid encoding a selectable marker can encode, for example, a β-lactamase, a luciferase, a green fluorescent protein (GFP), β-galactosidase, or other reporter gene as that term is understood in the art, including cell-surface markers, such as CD4 or the truncated nerve growth factor (NGFR) (for GFP, see WO 96/23810; Heim et al., Current Biology 2:178-182 (1996); Heim et al., Proc. Natl. Acad. Sci. USA (1995); or Heim et al., Science 373:663-664 (1995); for β-lactamase, see WO 96/30540). In a preferred embodiment, the selectable marker is a β-lactamase. The nucleic acid encoding a selectable marker can encode, for example, a fluorescent polypeptide. A fluorescent polypeptide can be detected by determining the amount of any quantitative fluorescent property, e.g., the amount of fluorescence at a particular wavelength, or the integral of fluorescence over an emission spectrum Optimally, the fluorescent polypeptide is selected to have fluorescent properties that are easily detected. Techniques for measuring fluorescence are well-known to one of skill in the art.

In the at least one nucleotide sequence encoding a gene product of interest for expression in a mammalian cell, the nucleotide sequence(s) is/are operably linked to at least one mammalian cell-compatible expression control sequence, e.g., a promoter. Many such promoters are known in the art. It will be understood by a skilled artisan that preferred promoters include those that are inducible, tissue-specific, or cell cycle-specific. For example, it was reported that the E2F promoter can mediate tumor-selective, and, in particular, neurological cell tumor-selective expression in vivo by being de-repressed in such cells in vivo. Parr et al., Nat. Med. 3:1145-9 (1997).

Applications of the Invention

A further aspect of the invention is a method of delivering a nucleotide sequence to a cell using the viral vectors, packaging functions, and helper functions described herein. The viral vector may be delivered to a cell in vitro or to a subject in vivo by any suitable method known in the art. Alternatively, the viral vector may be delivered to a cell ex vivo, and the cell administered to a subject, as known in the art.

The inventive methods and viral vectors may also be advantageously used in the treatment of individuals with metabolic disorders (e.g., omithine transcarbamylase deficiency). Duplexed vectors are preferred in such uses. Such disorders typically require a relatively rapid onset of expression of a therapeutic polypeptide by the packaged viral vector. As still a further alternative, the viral vectors may be administered to provide agents that improve transplant survivability (e.g., superoxide dismutase) or combat sepsis.

Moreover, dendritic cells (DC), which are refractory to wtAAV vectors (Jooss et al., (1998) 72:4212), are permissive for the viral vectors disclosed herein. Accordingly, as yet a further aspect, the viral vectors provide methods of delivering a nucleotide sequence to DC, e.g., to induce an immune response to a polypeptide encoded by the nucleotide sequence. Preferably, the nucleotide sequence encodes an antigen from an infectious agent or a cancer antigen.

As still a further aspect, viral vectors may be employed to deliver a heterologous nucleotide sequence in situations in which it is desirable to regulate the level of transgene expression (e.g., transgenes encoding hormones or growth factors, as described below). The more rapid onset of transgene expression by the viral vectors disclosed herein makes these gene delivery vehicles more amenable to such treatment regimes than are rAAV vectors.

Any heterologous nucleotide sequence(s) (as defined above) may be delivered by the viral vectors. Nucleic acids of interest include nucleic acids encoding polypeptides, preferably therapeutic (e.g., for medical or veterinary uses) or immunogenic (e.g., for vaccines) polypeptides.

Preferably, the heterologous nucleotide sequence or sequences will be less than about 2.5 kb in length (more preferably less than about 2.4 kb, still more preferably less than about 2.2 kb, yet more preferably less than about 2.0 kb in length) to facilitate packaging of the duplexed template by the parvovirus (e.g., AAV) capsid. Exemplary nucleotide sequences encode Factor IX, Factor X, lysosomal enzymes (e.g., hexosaminidase A, associated with Tay-Sachs disease, or iduronate sulfatase, associated, with Hunter Syndrome/MPS II), erythropoietin, angiostatin, endostatin, superoxide dismutase, globin, leptin, catalase, tyrosine hydroxylase, as well as cytokines (e.g., a interferon, β-interferon, interferon-γ, interleukin-2, interleukin-4, interleukin 12, granulocyte-macrophage colony stimulating factor, lymphotoxin, and the like), peptide growth factors and hormones (e.g., somatotropin, insulin, insulin-like growth factors 1 and 2, platelet derived growth factor, epidermal growth factor, fibroblast growth factor, nerve growth factor, neurotrophic factor-3 and 4, brain-derived neurotrophic factor, glial derived growth factor, transforming growth factor-α and -β, and the like), receptors (e.g., tumor necrosis factor receptor). In other exemplary embodiments, the heterologous nucleotide sequence encodes a monoclonal antibodies, preferably a single-chained monoclonal antibody or a monoclonal antibody directed against a cancer or tumor antigen (e.g., HER2/neu, and as described below). Other illustrative heterologous nucleotide sequences encode suicide gene products (thymdine kinase, cytosine deaminase, diphtheria toxin, cytochrome P450, deoxycytidine kinase, and tumor necrosis factor), proteins conferning resistance to a drug used in cancer therapy, and tumor suppressor gene products.

As a further alternative, the transgenes may encode a reporter polypeptide (e.g., an enzyme such as Green Fluorescent Protein, alkaline phosphatase).

Alternatively, in particular embodiments of the invention, the nucleic acid of interest may encode an antisense nucleic acid, a ribozyme (e.g., as described in U.S. Pat. No. 5,877,022), RNAs that affect spliceosome-mediated trans-splicing (see, Puttaraju et al., (1999) Nature Biotech. 17:246; U.S. Pat. No. 6,013,487; U.S. Pat. No. 6,083,702), interfering RNAs (RNAi) that mediate gene silencing (see, Sharp et al., (2000) Science 287:2431) or other non-translated RNAs, such as “guide” RNAs (Gorman et al., (1998) Proc. Nat. Acad. Sci. USA 95:4929; U.S. Pat. No. 5,869,248 to Yuan et al.), and the like.

The virus vector may also encode a heterologous nucleotide sequence that shares homology with and recombines with a locus on the host chromosome. This approach may be utilized to correct a genetic defect in the host cell.

The present invention may also be used to express an immunogenic polypeptide in a subject, e.g., for vaccination. The nucleic acid may encode any immunogen of interest known in the art including, but are not limited to, immunogens from human immunodeficiency virus, influenza virus, gag proteins, tumor antigens, cancer antigens, bacterial antigens, viral antigens, and the like.

The use of parvoviruses as vaccines is known in the art (see, e.g., Miyamura et al., (1994) Proc. Nat. Acad. Sci USA 91:8507; U.S. Pat. Nos. 5,916,563 to Young et al., 5,905,040 to Mazzara et al., U.S. Pat. No. 5,882,652, U.S. Pat. No. 5,863,541 to Samulski et al.; the disclosures of which are incorporated herein in their entirety by reference). The antigen may be presented in the parvovirus capsid. Alternatively, the antigen may be expressed from a heterologous nucleic acid introduced into a recombinant vector genome. Any immunogen of interest may be provided by the parvovirus vector. Immunogens of interest are well-known in the art and include, but are not limited to, immunogens from human immunodeficiency virus, influenza virus, gag proteins, tumor antigens, cancer antigens, bacterial antigens, viral antigens, and the like.

An immunogenic polypeptide, or immunogen, may be any polypeptide suitable for protecting the subject against a disease, including but not limited to microbial, bacterial, protozoal, parasitic, and viral diseases. For example, the immunogen may be an orthomyxovirus immunogen (e.g., an influenza virus immunogen, such as the influenza virus hemagglutinin (HA) surface protein or the influenza virus nucleoprotein gene, or an equine influenza virus immunogen), or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env genes products). The immunogen may also be an arenavirus immunogen (e.g., Lassa fever virus immunogen, such as the Lassa fever virus nucleocapsid protein gene and the Lassa fever envelope glycoprotein gene), a poxvirus immunogen (e.g., vaccinia, such as the vaccinia L1 or L8 genes), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP genes), a bunyavirus immunogen (e.g., RVFV, CCHF, and SFS viruses), or a coronavirus immunogen (e.g., an infectious human coronavirus immunogen, such as the human coronavirus envelope glycoprotein gene, or a porcine transmissible gastroenteritis virus immunogen, or an avian infectious bronchitis virus immunogen). The immunogen may further be a polio immunogen, herpes antigen (e.g., CMV, EBV, HSV immunogens) mumps immunogen, measles immunogen, rubella immunogen, diptheria toxin or other diptheria immunogen, pertussis antigen, hepatitis (e.g., hepatitis A or hepatitis B) immunogen, or any other vaccine immunogen known in the art.

Alternatively, the immunogen may be any tumor or cancer cell antigen. Preferably, the tumor or cancer antigen is expressed on the surface of the cancer cell. Exemplary cancer and tumor cell antigens are described in S. A. Rosenberg, (1999) Immunity 10:281). Other illustrative cancer and tumor antigens include, but are not limited to: BRCA1 gene product, BRCA2 gene product, gp100, tyrosinase, GAGE-1/2, BAGE, RAGE, NY-ESO-1, CDK4, β-catenin, MUM-1, Caspase-8, KIAA0205, HPVE, SART-1, PRAME, p15, melanoma tumor antigens (Kawakami et al., (1994) Proc. Natl. Acad. Sci. USA 91:3515); Kawakami et al., (1994) J. Exp. Med., 180:347); Kawakami et al., (1994) Cancer Res. 54:3124), including MART-1 (Coulie et al., (1991) J. Exp. Med. 180:35), gp100 (Wick et al., (1988) J. Cutan. Pathol. 4:201) and MAGE antigen, MAGE-1, MAGE-2 and MAGE-3 (Van der Bruggen et al., (1991) Science, 254:1643); CEA, TRP-1, TRP-2, P-15 and tyrosinase (Brichard et al., (1993) J. Exp. Med. 178:489); HER-2/neu gene product (U.S. Pat. No. 4,968,603), CA 125, LK26, FB5 (endosialin), TAG 72, AFP, CA19-9, NSE, DU-PAN-2, CASO, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (international patent publication WO 90/05142); telomerases; nuclear matrix proteins; prostatic acid phosphatase; papilloma virus antigens; and antigens associated with the following cancers: melanomas, metastases, adenocarcinoma, thymoma, lymphoma, sarcoma, lung cancer, liver cancer, colon cancer, non-Hodgkins lymphoma, Hodgkins lymphoma, leukemias, uterine cancer, breast cancer, prostate cancer, ovarian cancer, cervical cancer, bladder cancer, kidney cancer, pancreatic cancer and others (see, e.g., Rosenberg, (1996) Ann. Rev. Med. 47:481-91).

The heterologous nucleotide sequence may encode any polypeptide that is desirably produced in a cell in vitro, ex vivo, or in vivo. For example, viral vectors may be introduced into cultured cells and the expressed gene product isolated therefrom.

It will be understood by those skilled in the art that the heterologous nucleotide sequence(s) of interest may be operably associated with appropriate control sequences. For example, the heterologous nucleic acid may be operably associated with expression control elements, such as transcription/translation control signals, origins of replication, polyadenylation signals, and internal ribosome entry sites (IRES), promoters, enhancers, and the like.

Those skilled in the art will appreciate that a variety of promoter/enhancer elements may be used depending on the level and tissue-specific expression desired. The promoter/enhancer may be constitutive or inducible, depending on the pattern of expression desired. The promoter/enhancer may be native or foreign and can be a natural or a synthetic sequence. By foreign, it is intended that the transcriptional initiation region is not found in the wild-type host into which the transcriptional initiation region is introduced.

Promoter/enhancer elements that are native to the target cell or subject to be treated are most preferred. Also preferred are promoters/enhancer elements that are native to the transgene. The promoter/enhancer element is chosen so that it will function in the target cell(s) of interest. Mammalian or insect promoter/enhancer elements are also preferred. The promoter/enhance element may be constitutive or inducible.

Inducible expression control elements are preferred in those applications in which it is desirable to provide regulation over expression of the transgene(s). Inducible promoters/enhancer elements for gene delivery are preferably tissue-specific promoter/enhancer elements, and include muscle specific (including cardiac, skeletal and/or smooth muscle), neural tissue specific (including brain-specific), liver specific, bone marrow specific, pancreatic specific, spleen specific, retinal specific, and lung specific promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metalothionein promoter.

In embodiments of the invention in which the transgene(s) will be transcribed and then translated in the target cells, specific initiation signals are generally required for efficient translation of inserted protein coding sequences. These exogenous translational control sequences, which may include the ATG initiation codon and adjacent sequences, can be of a variety of origins, both natural and synthetic.

The methods of the present invention also provide a means for delivering heterologous nucleotide sequences into a broad range of cells, including dividing and non-dividing cells. The present invention may be employed to deliver a nucleotide sequence of interest to a cell in vitro, e.g., to produce a polypeptide in vitro or for ex vivo gene therapy. The cells, pharmaceutical formulations, and methods of the present invention are additionally useful in a method of delivering a nucleotide sequence to a subject in need thereof, e.g., to express an immunogenic or therapeutic polypeptide. In this manner, the polypeptide may thus be produced in vivo in the subject. The subject may be in need of the polypeptide because the subject has a deficiency of the polypeptide, or because the production of the polypeptide in the subject may impart some therapeutic effect, as a method of treatment or otherwise, and as explained further below.

In general, the present invention may be employed to deliver any foreign nucleic acid with a biological effect to treat or ameliorate the symptoms associated with any disorder related to gene expression. Illustrative disease states include, but are not-limited to: cystic fibrosis (and other diseases of the lung), hemophilia A, hemophilia B, thalassemia, anemia and other blood disorders, AIDs, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Gauchers disease, Hurler's disease, adenosine deaminase deficiency, glycogen storage diseases and other metabolic defects, retinal degenerative diseases (and other diseases of the eye), diseases of solid organs (e.g., brain, liver, kidney, heart), and the like.

Gene transfer has substantial potential use in understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer could be used to bring a normal gene into affected tissues for replacement therapy, as well as to create animal models for the disease using antisense mutations. For unbalanced disease states, gene transfer could be used to create a disease state in a model system, which could then be used in efforts to counteract the disease state. Thus the methods of the present invention permit the treatment of genetic diseases. As used herein, a disease state is treated by partially or wholly remedying the deficiency or imbalance that causes the disease or makes it more severe. The use of site-specific recombination of nucleic sequences to cause mutations or to correct defects is also possible.

The instant invention may also be employed to provide an antisense nucleic acid to a cell in vitro or in vivo. Expression of the antisense nucleic acid in the target cell diminishes expression of a particular protein by the cell. Accordingly, antisense nucleic acids may be administered to decrease expression of a particular protein in a subject in need thereof. Antisense nucleic acids may also be administered to cells in vitro to regulate cell physiology, e.g., to optimize cell or tissue culture systems.

Finally, the instant invention finds further use in diagnostic and screening methods, whereby a transgene is transiently or stably expressed in a cell culture system, or alternatively, a transgenic animal model.

In general, the present invention can be employed to deliver any heterologous nucleic acid to a cell in vitro, ex vivo, or in vivo.

Subjects, Pharmaceutical Formulations, Vaccines, and Modes of Administration

The present invention finds use in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

In particular embodiments, the present invention provides a pharmaceutical composition comprising a virus particle of the invention in a pharmaceutically-acceptable carrier and/or other medicinal agents, pharmaceutical agents, carriers, adjuvants, diluents, etc. For injection, the carrier will typically be a liquid. For other methods of administration, the carrier may be either solid or liquid. For inhalation administration, the carrier will be respirable, and will preferably be in solid or liquid particulate form. As an injection medium, it is preferred to use water that contains the additives usual for injection solutions, such as stabilizing agents, salts or saline, and/or buffers.

Exemplary pharmaceutically acceptable carriers include sterile, pyrogen-free water and sterile, pyrogen-free, phosphate buffered saline. Physiologically-acceptable carriers include pharmaceutically-acceptable carriers. Pharmaceutically acceptable carriers are those which are that is not biologically or otherwise undesirable, i.e., the material may be administered to a subject without causing undesirable biological effects which outweigh the advantageous biological effects of the material.

A pharmaceutical composition may be used, for example, in transfection of a cell ex vivo or in administering a viral particle or cell directly to a subject.

The parvovirus vectors of the invention may be administered to elicit an immunogenic response (e.g., as a vaccine). Typically, vaccines of the present invention comprise an immunogenic amount of infectious virus particles as disclosed herein in combination with a pharmaceutically-acceptable carrier. An “immunogenic amount” is an amount of the infectious virus particles that is sufficient to evoke an immune response in the subject to which the pharmaceutical formulation is administered. Typically, an amount of about 1 to about 10¹⁵ virus particles, preferably about 10⁴ to about 10¹⁰, and more preferably about 10⁴ to 10⁶ virus particles per dose is suitable, depending upon the age and species of the subject being treated, and the immunogen against which the immune response is desired. Subjects and immunogens are as described above.

The present invention further provides a method of delivering a nucleic acid to a cell. Typically, for in vitro methods, the virus may be introduced into the cell by standard viral transduction methods, as are known in the art. Preferably, the virus particles are added to the cells at the appropriate multiplicity of infection according to standard transduction methods appropriate for the particular target cells. Titers of virus to administer can vary, depending upon the target cell type and the particular virus vector, and may be determined by those of skill in the art without undue experimentation.

Recombinant virus vectors are preferably administered to the cell in a biologically-effective amount. If the virus is administered to a cell in vivo (e.g., the virus is administered to a subject as described below), a biologically-effective amount of the virus vector is an amount that is sufficient to result in transduction and expression of the transgene in a target cell.

The cell to be administered the inventive virus vector may be of any type, including but not limited to neural cells (including cells of the peripheral and central nervous systems, in particular, brain cells), lung cells, retinal cells, epithelial cells (e.g., gut and respiratory epithelial cells), muscle cells, dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. Alternatively, the cell may be any progenitor cell. As a further alternative, the cell can be a stem cell (e.g., neural stem cell, liver stem cell). As still a further alternative, the cell may be a cancer or tumor cell. Moreover, the cells can be from any species of origin, as indicated above.

In particular embodiments of the invention, cells are removed from a subject, a parvovirus vector is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, an rAAV vector is introduced into cells from another subject, into cultured cells, or into cells from any other suitable source, and the cells are administered to a subject in need thereof.

The cells transduced with a viral vector are preferably administered to the subject in a “therapeutically-effective amount” in combination with a pharmaceutical carrier. Those skilled in the art will appreciate that the therapeutic effects need not be complete or curative, as long as some benefit is provided to the subject.

In alternate embodiments, cells that have been transduced with a vector according to the invention may be administered to elicit an immunogenic response against the delivered polypeptide. Typically, a quantity of cells expressing an immunogenic amount of the polypeptide in combination with a pharmaceutically-acceptable carrier is administered. An “immunogenic amount” is an amount of the expressed polypeptide that is sufficient to evoke an active immune response in the subject to which the pharmaceutical formulation is administered. The degree of protection conferred by the active immune response need not be complete or permanent, as long as the benefits of administering the immunogenic polypeptide outweigh any disadvantages thereof.

Dosages of the cells to administer to a subject will vary upon the age, condition and species of the subject, the type of cell, the nucleic acid being expressed by the cell, the mode of administration, and the like. Typically, at least about 10² to about 10⁸, preferably about 10³ to about 10⁸ cells, will be administered per dose. Preferably, the cells will be administered in a therapeutically-effective amount.

A further aspect of the invention is a method of treating subjects in vivo with the viral particles. Administration of the parvovirus particles of the present invention to a human subject or an animal in need thereof can be by any means known in the art for administering virus vectors.

Exemplary modes of administration include oral, rectal, transmucosal, topical, transdermal, inhalation, parenteral (e.g., intravenous, subcutaneous, intradermal, intramuscular, and intraarticular) administration, and the like, as well as direct tissue or organ injection, alternatively, intrathecal, direct intramuscular, intraventricular, intravenous, intraperitoneal, intranasal, or intraocular injections. Injectables can be prepared in conventional forms, either as liquid solutions or suspensions, solid forms suitable for solution or suspension in liquid prior to injection, or as emulsions. Alternatively, one may administer the virus in a local rather than systemic manner, for example, in a depot or sustained-release formulation.

The parvovirus vector administered to the subject may transduce any permissive cell or tissue. Suitable cells for transduction by the inventive parvovirus vectors are as described above.

In particularly preferred embodiments of the invention, the nucleotide sequence of interest is delivered to the liver of the subject. Administration to the liver may be achieved by any method known in the art, including, but not limited to intravenous administration, intraportal administration, intrabiliary administration, intra-arterial administration, and direct injection into the liver parenchyma.

In other preferred embodiments, the inventive parvovirus particles are administered intramuscularly, more preferably by intramuscular injection or by local administration (as defined above). Delivery to the brain is also preferred. In other preferred embodiments, the parvovirus particles of the present invention are administered to the lungs.

The parvovirus vectors disclosed herein may be administered to the lungs of a subject by any suitable means, but are preferably administered by administering an aerosol suspension of respirable particles comprised of the inventive parvovirus vectors, which the subject inhales. The respirable particles may be liquid or solid. Aerosols of liquid particles comprising the inventive parvovirus vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the inventive virus vectors may likewise be produced with any solid particulate medicament aerosol generator, by techniques known in the pharmaceutical art.

Dosages of the inventive parvovirus particles will depend upon the mode of administration, the disease or condition to be treated, the individual subject's condition, the particular viral vector, and the gene to be delivered, and can be determined in a routine manner. Exemplary doses for achieving therapeutic effects are virus titers of at least about 10⁵, 10⁶, 10⁷, 10⁸, 10⁹, 10¹⁰, 10¹¹, 10¹², 10¹³, 10¹⁴, 10¹⁵ transducing units or more, preferably about 10⁸-10¹³ transducing units, yet more preferably 10¹² transducing units.

In particular embodiments, the parvovirus particles are administered as part of a method of treating cancer or tumors by administering anti-cancer agents (e.g., cytokines) or a cancer or tumor antigen. The parvovirus particle may be administered to a cell in vitro or to a subject in vivo or by using ex vivo methods, as described herein and known in the art.

The term “cancer” has its understood meaning in the art, for example, an uncontrolled growth of tissue that has the potential to spread to distant sites of the body (i.e., metastasize). Exemplary cancers include, but are not limited to, leukemias, lymphomas, colon cancer, renal cancer, liver cancer, breast cancer, lung cancer, prostate cancer, ovarian cancer, melanoma, and the like. Preferred are methods of treating and preventing tumor-forming cancers. The term “tumor” is also understood in the art, for example, as an abnormal mass of undifferentiated cells within a multicellular organism. Tumors can be malignant or benign. Preferably, the inventive methods disclosed herein are used to prevent and treat malignant tumors.

Cancer and tumor antigens have been described hereinabove. By the terms “treating cancer” or “treatment of cancer”, it is intended that the severity of the cancer is reduced or the cancer is at least partially eliminated. Preferably, these terms indicate that metastasis of the cancer is reduced or at least partially eliminated. It is further preferred that these terms indicate that growth of metastatic nodules (e.g., after surgical removal of a primary tumor) is reduced or at least partially eliminated. By the terms “prevention of cancer” or “preventing cancer” it is intended that the inventive methods at least partially eliminate or reduce the incidence or onset of cancer. Alternatively stated, the present methods slow, control, decrease the likelihood or probability, or delay the onset of cancer in the subject.

Likewise, by the terms “treating tumors” or “treatment of tumors”, it is intended that the severity of the tumor is reduced or the tumor is at least partially eliminated. Preferably, these terms are intended to mean that metastasis of the tumor is reduced or at least partially eliminated. It is also preferred that these terms indicate that growth of metastatic nodules (e.g., after surgical removal of a primary tumor) is reduced or at least partially eliminated. By the terms “prevention of tumors” or “preventing tumors” it is intended that the inventive methods at least partially eliminate or reduce the incidence or onset of tumors. Alternatively stated, the present methods slow, control, decrease the likelihood or probability, or delay the onset of tumors in the subject.

In other embodiments, cells may be removed from a subject with cancer or a tumor and contacted with the parvovirus particles of the invention. The modified cell is then administered to the subject, whereby an immune response against the cancer or tumor antigen is elicited. This method is particularly advantageously employed with immunocompromised subjects that cannot mount a sufficient immune response in vivo (i.e., cannot produce enhancing antibodies in sufficient quantities).

It is known in the art that immune responses may be enhanced by immunomodulatory cytokines (e.g., α-interferon, β-interferon, γ-interferon, o-interferon, τ-interferon, interleukin-1α, interleukin-1β, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-18, B cell Growth factor, CD40 Ligand, tumor necrosis factor-β, tumor necrosis factor-α, monocyte chemoattractant protein-1, granulocyte-macrophage colony stimulating factor, and lymphotoxin). Accordingly, in particular embodiments of the invention, immunomodulatory cytokines (preferably, CTL inductive cytokines) are administered to a subject in conjunction with the methods described herein for producing an immune response or providing immunotherapy.

Cytokines may be administered by any method known in the art. Exogenous cytokines may be administered to the subject, or alternatively, a nucleotide sequence encoding a cytokine may be delivered to the subject using a suitable vector, and the cytokine produced in vivo.

Having now described the invention, the same will be illustrated with reference to certain examples, which are included herein for illustration purposes only, and which are not intended to be limiting of the invention.

### Examples

### Materials & Methods

Construction of Baculoviral Shuttle Plasmids

Baculoviral shuttle plasmids LSR (expressing Rep 78/52), VPm11 (expressing VP1, VP2, and VP3), and GFPR (AAV ITRs flanking reporter gene GFP) were kindly provided by Dr. Robert Kotin from NIH (see Kotin et al., US Patent Publication No. 2004/0197895, the entire disclosure of which is incorporated herein by reference). Baculoviral shuttle plasmid pFB-2.5Cap was constructed by cloning the EcoNI-NotI fragment of 2.5Cap gene from pXR2.5 into the EcoNI-NotI sites of VPm11. Plasmid pFB-2.5VP1 was constructed by cloning the SwaI-NotI fragment from pxr2.5 into the NotI & Klenow-blunt-ended BamHI sites of VPm11.

Generation of Recombinant Baculoviruses

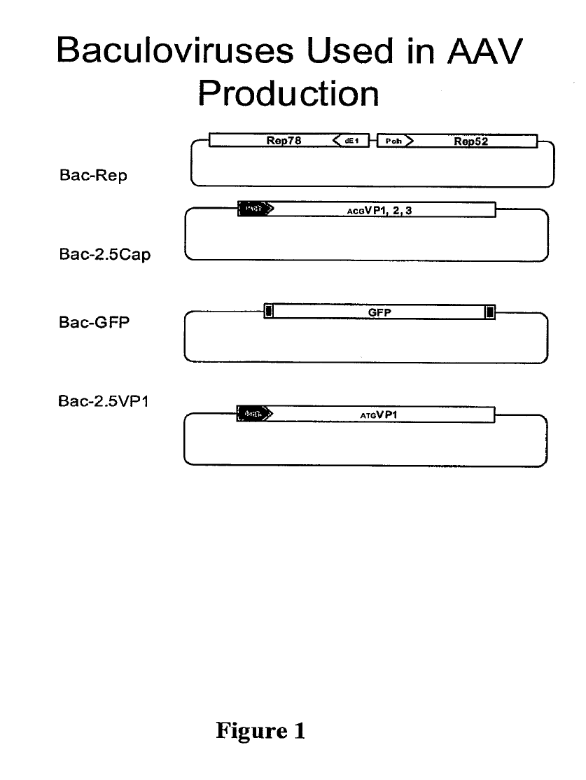

Recombinant Baculoviruses were generated according to the Bac-to-bac protocols of Invitrogen with modifications. Briefly, 2 ng of shuttle plasmid was transformed into 20 μl of DH10Bac competent cells and several white colonies were picked after 48-hour incubation and miniprep bacmid DNAs were prepared. The miniprep DNAs were then transfected into Sf9 cells using CellFectine in 6-well plates to generate recombinant Baculoviruses. The recombinant Baculoviruses were harvested after 3-day transfection period and amplified. The amplified Baculoviruses were used for titration and subsequent AAV production. The recombinant baculoviruses used in this study are diagramed in FIG. 1.

Cell Cultures

293 cells were maintained in DMEM media supplemented with 10% FBS and 100 units/ml of penicillin and 100 μg/ml of streptomycin. The cells were passaged twice a week. HepG2 cells were maintained in MEME media (ATCC) supplemented with 10% FBS and 100 units/ml of penicillin and 100 μg/ml of streptomycin. The cells were passaged once a week. Sf9 cells were maintained as suspension culture in shaker flasks in SF900II or ExCell420 media supplemented with 100 units/ml of penicillin and 100 μg/ml of streptomycin. The cells were passaged twice a week.

Baculovirus Titration

The recombinant Baculoviruses were titrated using the rapid titer kit according to the protocols of manufacturer (BD BIOSCIENCES). Briefly, Sf9 cells were seeded in 96-well plate at 6.5⁺⁴ cells/well for one hour and serial diluted baculovirus solution was added to infect the cells for one hour. The baculovirus solution was then removed and methyl cellulose containing culture media was added. After 48-hour incubation, infection loci were detected by probing with gp64 antibody in color matrix reaction.

AAV Vector Production, Purification, and Titration

To produce AAV vectors in Sf9 cells with the shaker flasks or Wave bioreactor, the cells were first grown to about 5E+6 cells/ml in SF90011 or ExCell420 media supplemented with 100 units/ml of penicillin and 100 μg/ml of streptomycin. Right before the infection, another half of fresh media mixed with required amounts of recombinant baculoviruses were added to the cell culture to bring the cell number to about 2.5E+6 cells/ml. The infection was carried out for 3 days and cell pellets were harvested by centrifugation at 3,000 rpm for 10 min. The cell pellets were lysed in Sf9 lysis buffer (1% DOC, 0.5% CHAPS, 50 mM NaCl, 2 mM MgCl₂, 50 mM Tris-HCl, pH8.0). Benzonase at 125 units/ml was added to digest the genomic DNA by incubating at 37° C. for 1 hour. The salt concentration was adjusted to 400 mM after the incubation and cell debris was removed by centrifugation at 8,000 rpm for 30 min. The cleared lysates were loaded onto the step CsCl-gradient and subjected to 2 rounds of ultracentrifugation. AAV vectors were harvested and dialysed against 100 volumes of PBS containing 5% sorbitol and used for subsequent experiments.

Dot Blot Analysis of AAV Titers

Purified AAV vectors were first digested with DNase (10 mM Tris pH7.5, 10 mM MgCl₂, 50 units/ml DNase 1) for 1 hour at 37° C. to remove any contaminated DNA and then the digestion was stopped by adding EDTA to 20 mM. The viral DNA was released by digestion at 50° C. with equal volume of Proteinase K (1M NaCl, 100 ug/ml proteinase K, 1% sarkosyl) for two hours and proteins removed by phenol/chloroform extraction. The viral DNA was then precipitated and resuspended in TE buffer. Viral copy number was determined by hybridizing with radioactive labeled DNA probes using dot-blot apparatus.

Coommassie Blue Staining

Purified AAV vectors were boiled in sample buffer for 5 min and capsid proteins separated by SDS-PAGE. The gels were then fixed in fixation solution containing 25% isopropanol, 10% acetic acid, and 65% milli-Q water for 20 min and stained overnight in staining solution containing 0.01% R-250 Coomassie (BioRad) and 10% acetic acid with gentle shaking. The gels were destained in destaining solution containing 10% acetic acid with several solution changes until the background was clear.

Western Blot and Silver-Staining

Cell lysates or AAV vectors were boiled in 1×SDS sample buffer for 5 min and the boiled samples were subjected to SDS-PAGE. For Western blot, the proteins separated on the gels were transferred onto nitrocellulose membranes. The membranes were blocked by 5% skim milk and probed with anti-AAV VP monoclonal antibody (B1 clone) followed by HRP-cojugated anti-mouse monoclonal antibody. The signals were captured on film using the SuperSignal West Femto Maximum Sensitivity Substrate (PIERCE). For silver-staining, the gels were stained according to the manufacturer's protocol using the SilverXpress kit (Invitrogen).

Transduction of 293 and HepG2 Cells

The cells were seeded at 1.5×10⁵ cells/well in 24-well plates one day before transduction. AAV vectors were serial diluted into 10⁻¹, 10⁻², 10⁻³, and 10⁻⁴ in 1 ml of media containing 1.5 μM (for 293 cells) or 20 μM (for HepG2 cells) of etoposide. Old media were removed from the cells and 0.5 ml of diluted AAV was added. The cells were cultured for 48 hours and GFP expressing cells were counted under fluorescent microscope.

### Examples

### Results

Sf9 cell produced AAV2.5-GFP vectors have lower VP1/VP3 ratio than those produced by 293 cells

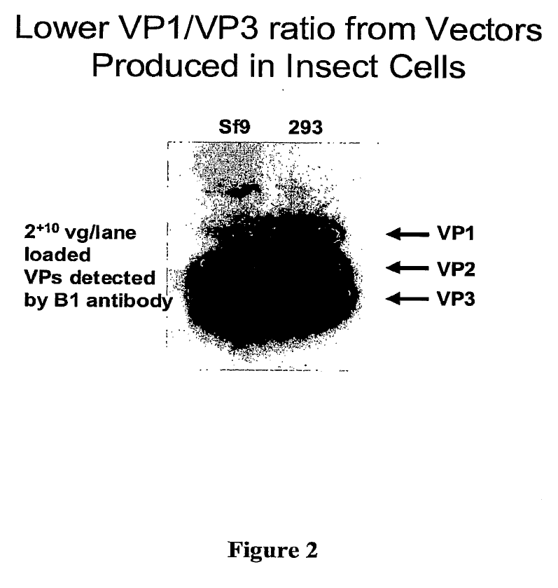

AAV2.5-GFP vectors produced in Sf9 cells were compared with their 293 counterparts to check the capsid composition by Western blot analyses. FIG. 2 shows that Sf9 cell packaged viral particles have lower VP1/VP3 ratio than those packaged in 293 cells, which indicates that Sf9 cells may package some VP1-viral particles. This result was also confirmed by Coomassie staining and silver staining analyses (data not shown).

Sf9 cell produced AAV2.5-GFP vectors have much lower infectivity than those produced by 293 cells

To compare the infectivity of Sf9 and 293 cell produced AAV2.5-GFP vectors, HepG2 cells were transduced by the vectors for 48 hours and green cell numbers were counted. The results in Table 1 show that AAV2.5-GFP vectors produced in Sf9 cells have much lower infectivity as compared with their 293 counterpart. Similar results were observed when 293 cells were used for the transduction (data not shown).

Adding extra 2.5VP1 increases the VP1/VP3 ratio of AAV vectors produced in Sf9 cells

Different amounts of 2.5VP1 were introduced by co-infecting the Sf9 cells with 0.1, 0.5, and 1.0 moi of the 2.5VP1 expressing baculovirus (Bac-2.5VP1) together with 1 moi of each of the other three baculoviruses (Bac-Rep, Bac-2.5Cap, and Bac-GFP) for AAV packaging. After 3 days of infection, cells were harvested. A small part of the cells was used to prepare cell lysates and majority of the cell pellets were used for AAV purification. Both the lysates and purified AAV vectors were subjected to SDS-PAGE and capsid proteins detected by Western blot method. The results shown below in Table 1 indicate that VP1 expression levels were increased and correlated with the increased amounts of VP1 baculoviruses.

| AAV Lot# | Vector type | Cell source | Relative infectivity (%) |
|---|---|---|---|
| 018 | AAV2.5-GFP | Sf9 | 0.0008 |
| 019 | AAV2.5-GFP | Sf9 | 0.0072 |
| 020 | AAV2.5-GFP | Sf9 | 0.0016 |
| 293 | AAV2.5-GFP | 293 | 100 |

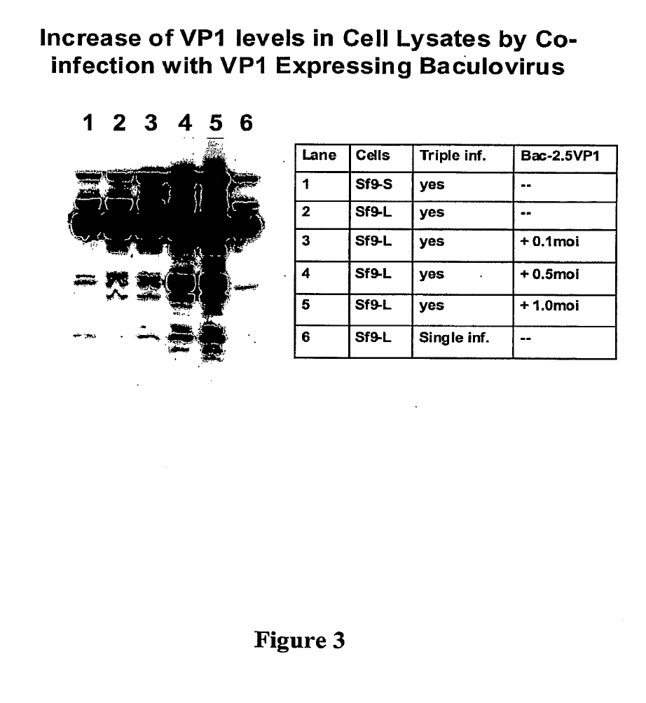

At the same time it was observed that when more VP1 was expressed, there were more degraded capsid proteins. Purified AAV particles also contain more VP1 when more Bac-2.5VP1 was used (FIG. 3).

Increase of VPlNP3 ratio improves the infectivity of AAV vectors produced in Sf9 cells

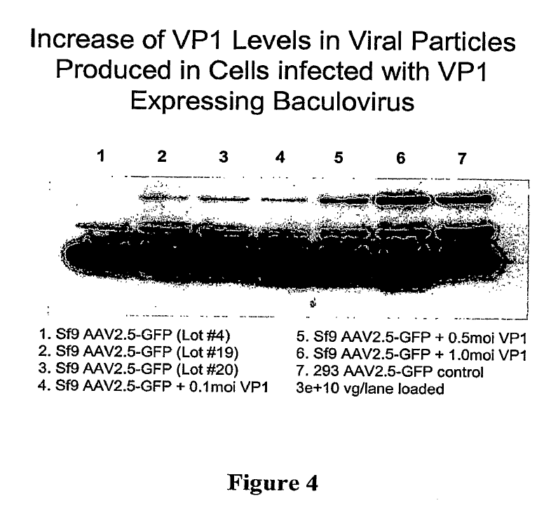

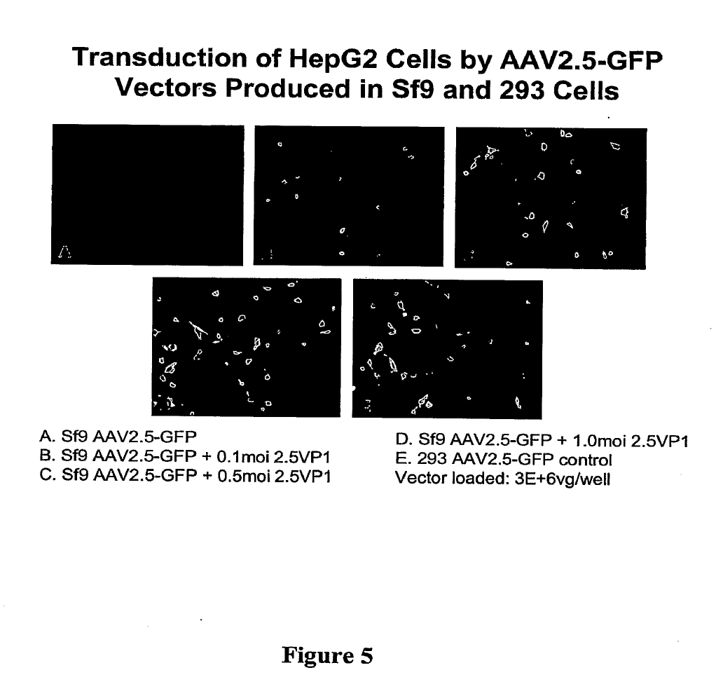

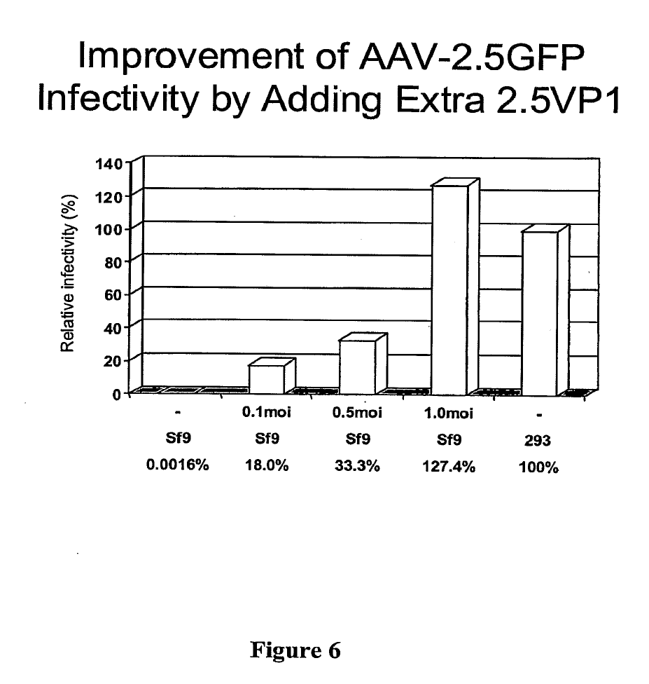

The increase of VP1/VP3 ratio improves the infectivity of AAV vectors produced in Sf9 cells. Since VP1 contains a phospholipase domain that is required for AAV infectivity, we tested if adding extra VP1 during the vector production process could improve the infectivity of AAV vectors produced in Sf9 cells. The AAV vectors were used to transduce HepG2 cells for 48 hours and green cells were scored and photographed. The results in FIGS. 4-6 indicate that the infectivity of the AAV2.5-GFP vectors was dramatically improved by adding extra VP1.

Alternative Recombinant Baculovirus Vectors

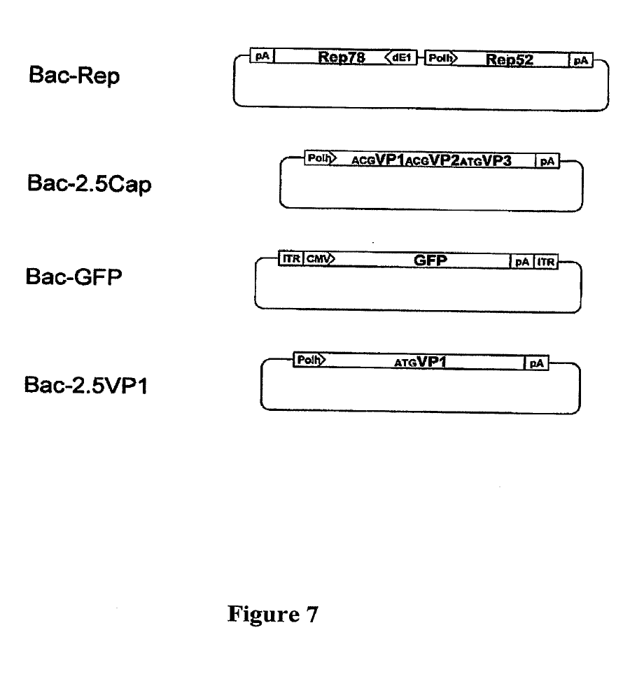

FIGS. 7-11 illustrate alternative Bac vectors for producing infectious AAV viruses. For example, replication components can be combined in a single vector or included in separate vectors. Further genes expressing VP1, VP2 and VP3 can be included in a Cap vector with or without the additional VP1 gene for increase expressed amounts of VP1 viral particles. Further, the transgene, flanked by TRs, can be exclusively included in a single Bac vector or combined with other components such as the gene for expressing VP1, replication component(s) and/or genes expressing VP1, VP2 and VP3.

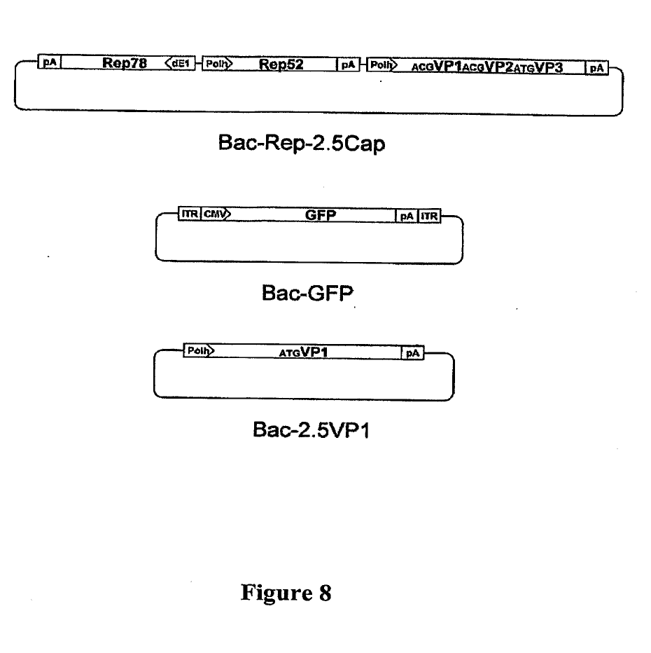

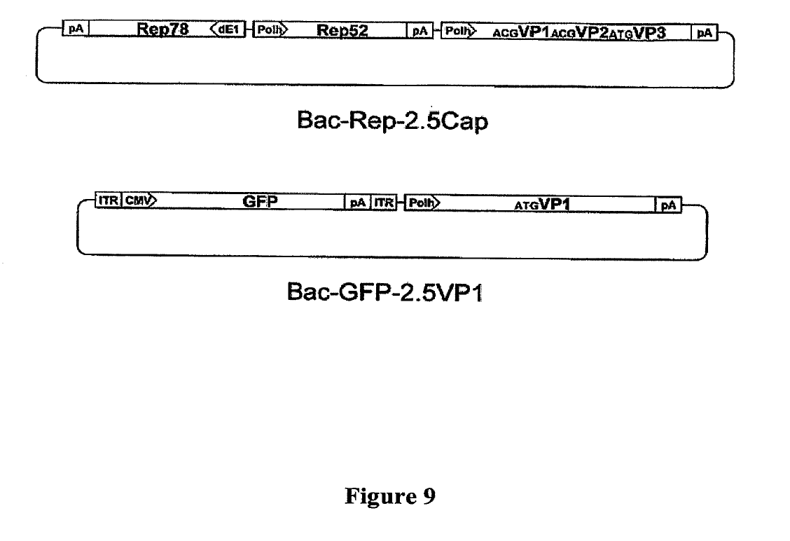

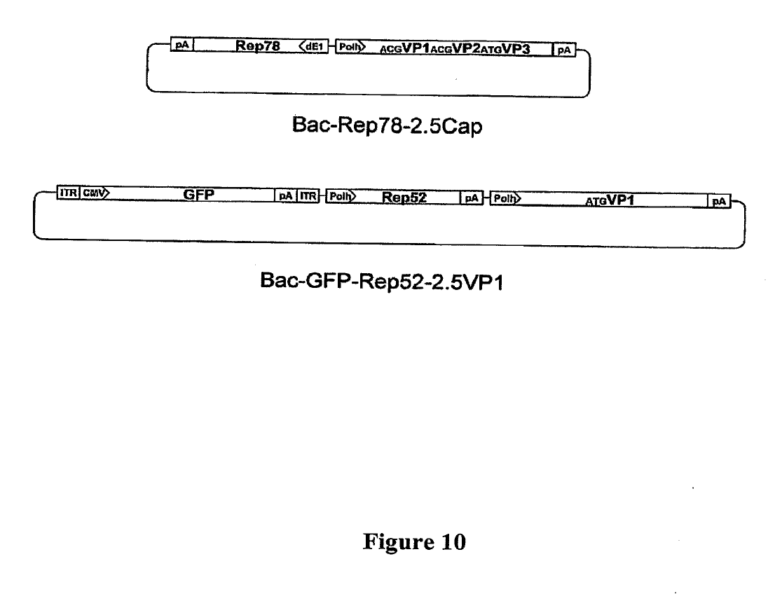

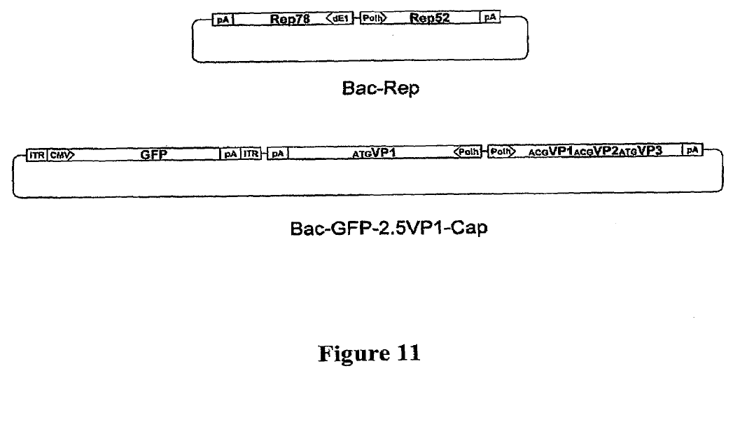

FIG. 7 illustrates four additional recombinant baculoviruses that may be used together in generating infectious viral particles, wherein the vector comprising GFP or transgene under the control of a cytomegalovirus (CMV) immediate early promoter/enhancer and a poly-A sequence generally inserted following the transgene sequence and before the 3′ AAV ITR sequence. Separate Bac-vectors provided for additional and necessary genes, including VP1 gene using the original ATG start codon with polh promoter, a vector with the Cap gene and another vector comprising the Rep78 and 52 genes. Notably, all vectors include at least one polyadenylation sequence (pA). FIG. 8 illustrates a system using three Bac vectors, wherein one Bac vector includes the 2.5 Cap gene encoding VP1, VP2 and VP3 and AAV replication components. Other vectors include one for expression of 2.5 VP1 and another for expression of the transgene. FIG. 9 shows the use of only two vectors, wherein the 2.5 Cap gene encoding VP1, VP2 and VP3 is included in the same vector with AAV replication components, and the second vector includes genes for GFP (transgene) and the 2.5 VP1 gene for expressing additional amounts of VP1. FIG. 10 also includes a system using only two vectors, wherein a single replication component is included in each Bac vector, and advantageously, the Rep 78 is in a separate vector from the transgene, thereby increasing stability of vector and subsequent expression. Further in one vector, the transgene and the 2.5 VP1 gene are combined with a Rep gene. FIG. 11 also shows two vectors, wherein two replication components are included in a single Bac vector and the 2.5 Cap gene encoding VP1, VP2 and VP3 are included in the same vector as the additional 2.5 VP1 gene and transgene.

Although the foregoing invention has been described in some detail by way of illustration and example for purposes of clarity and understanding, it will be apparent that changes and modifications may be practiced within the scope of the appended claims and equivalents thereof.

What is claimed is:

1. A method of producing a packaged parvovirus vector, the method comprising:
(a) providing an insect cell;
(b) introducing into the insect cell one or more baculovirus vectors comprising nucleotide sequences encoding:
(i) a transgene flanked by TRs; and
(ii) baculovirus packaging functions and parvovirus Rep and Cap components sufficient to result in packaging of infective parvovirus particles, wherein VP1 is supplemented relative to VP2 and VP3 sufficient to increase the production of infectious viral particles; and
(c) introducing into the insect cell a nucleic acid encoding baculovirus helper functions for expression in the insect cell; and
(d) culturing the insect cell under conditions sufficient to produce the infectious packaged parvovirus vector.

2. The method of claim 1 wherein the VP1 supplementation is effected by:
(a) introducing into the insect cell a Cap vector comprising nucleotide sequences expressing VP1, VP2 and VP3 and a VP1 vector comprising nucleotide sequences expressing VP1;
(b) introducing into the insect cell a single vector comprising nucleotide sequences expressing VP1, VP2 and VP3 and additional nucleotide sequences expressing VP1;
(c) introducing into the insect cell a parvovirus Cap vector comprising optimized nucleotide sequences for expression of VP1, VP2 and/or VP3 in the insect cells; or
(d) introducing into the insect cell a VP1 vector comprising optimized nucleotide sequences for expression of VP1 in the insect cells.

3. The method of claim 1, wherein the parvovirus is Adeno-associated virus (AAV).

4. (canceled)

5. The method of claim 3 wherein expression of the VP1 vector is from about 1 to about 75% of the expression of the parvovirus Cap vector.

6. (canceled)

7-10. (canceled)

11. The method of claim 1 wherein the supplementation of VP1 results in production of a molecular ratio of approximately 10:10:80 VP1:VP2:VP3.

12. (canceled)

13. The method of claim 3 wherein the supplementation of VP1 results in production of infectious packaged parvovirus vectors in an amount which is about 10, 20, 30, 40, 50, 60, 70, 80, 90, 100, 110, 120, 130, 140, 15, 160, 170, 180, 190 or 200 times greater than in corresponding method in the absence of the supplementation.

14. The method of claim 1 wherein the nucleotide sequences comprise a duplexed vector, amplicon, and/or plasmid.

15-16. (canceled)

17. The method of claim 1 wherein one or more of the parvovirus Rep functions and parvovirus Cap functions are inserted into the same vector.

18. The method of claim 1, wherein the insect cell is a Lepidopteran cell or is derived from a Lepidopteran cell.

19. The method of claim 1, wherein the insect cell is a species selected from the group consisting of Spodoptera frugiperda and Trichopulsia ni.

20-21. (canceled)

22. The method of claim 1, wherein the parvovirus Cap functions are AAV-2 Cap functions comprising the following mutations: 263 Q→A; 265 insertion T; 705 N→A; 708 V→A; and 716 T→N (SEQ ID NO: 13).

23. The method of claim 1, wherein the transgene encodes a therapeutic product.

24. The method according to claim 1, wherein a first vector comprises at least one of the group consisting of the transgene flanked by TRs, a gene encoding AAV-VP1 operatively connected to a promoter, a gene encoding AAV VP1 VP2 and VP3, and a replication component.

25-26. (canceled)

27. The method according to claim 1, wherein a second vector comprises at least one replication component and a gene encoding AAV-VP1, VP2 and VP3.

28. (canceled)

29. The method of claim 3, wherein the AAV Cap component comprises an optimized nucleotide sequence for expression in the insect cell selected from the group consisting of: SEQ ID NO: 5; SEQ ID NO: 6; SEQ ID NO: 8; and SEQ ID NO: 10.

30. The method of claim 3, wherein the AAV Rep component comprises an optimized nucleotide sequence for expression in the insect cell selected from the group consisting of: SEQ ID NO: 2 and SEQ ID NO: 4.

31. The method of claim 3, comprising an optimized nucleotide sequence for expression in the insect cell selected from the group consisting of: SEQ ID NO: 5; SEQ ID NO: 6; SEQ ID NO: 8; SEQ ID NO: 10; SEQ ID NO: 2 and SEQ ID NO: 4.

32. The method of claim 3, wherein the AAV is AAV2, AAV 2.5, AAV 8 or AAV 9.

33. The method of claim 32, wherein the AAV comprises a nucleotide sequence selected from the group consisting of: SEQ ID NO: 1; SEQ ID NO: 3; SEQ ID NO: 5; SEQ ID NO: 11; SEQ ID NO: 7; and SEQ ID NO: 9.

34-36. (canceled)

37. A kit for producing packaged parvovirus vector, the kit comprising at least a first and second vector, wherein the vectors comprise nucleotide sequences encoding:
(i) a transgene flanked by TRs; and
(ii) baculovirus packaging functions comprising parvovirus Rep components and parvovirus Cap components sufficient to result in packaging of infective parvovirus particles, wherein VP1 is supplemented relative to VP2 and VP3 sufficient to increase the production of infectious viral particles.

38-48. (canceled)

49. A host cell comprising one or more vectors comprising nucleotide sequences encoding:
(i) a transgene flanked by TRs; and
(ii) baculovirus helper functions for the replication of AAV Rep components and AAV Cap components and packaging of infective parvovirus particles, wherein VP1 is supplemented relative to VP2 and VP3 sufficient to increase the production of infectious viral particles.

50-51. (canceled)


SEQUENCE LISTING (13 sequences)

SEQ ID NO: 1 - DNA, 1194 bp, adeno-associated virus 2
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 2 - DNA, 1243 bp, Artificial Sequence
  Synthetic Construct
gggcgaattgggtaccatcgatatggaactggtcggttggctggtcgacaagggtatcacctccgagaagcagtggatccaggaagatcaggcttcctacatctccttcaacgctgcttccaactcccgttcccagatcaaggctgctctggacaacgctggcaagatcatgtccctgaccaagaccgctcccgactacctggtcggccagcagcccgtggaggacatctcctccaaccgcatctacaagatcctcgagctgaacggttacgacccccagtacgctgcctccgtgttcctgggttgggctaccaagaagttcggcaagcgtaacaccatctggctgttcggtcccgctaccaccggcaagaccaacatcgctgaggctatcgctcacaccgtgcccttctacggttgcgtgaactggaccaacgagaacttccccttcaacgactgcgtggacaagatggtgatttggtgggaggaaggcaagatgaccgctaaggtggtcgagtccgctaaggctatcctgggcggttccaaggtccgcgtggaccagaagtgcaagtcctccgctcagatcgaccccacccccgtgatcgtgacctccaacaccaacatgtgcgctgtgatcgacggtaactccaccactttcgagcaccagcagcctctgcaggaccgtatgttcaagttcgagctgacccgtcgtctggaccacgacttcggcaaggtgaccaagcaggaagtgaaggacttcttccgttgggctaaggaccacgtggtggaggtggagcacgagttctacgtgaagaagggtggcgctaagaagcgtcccgctccctccgacgctgacatctccgagcccaagcgtgtgcgcgagtccgtggcccagccctccacctccgacgccgaggcttccatcaactacgctgaccgctaccagaacaagtgctcccgtcacgtgggcatgaacctgatgctgttcccttgccgtcagtgcgagcgtatgaaccagaactccaacatctgcttcacccacggccagaaggactgcctcgagtgcttccccgtgtccgagtcccagcccgtgtccgtggtgaagaaggcttaccagaagctgtgctacatccaccacatcatgggcaaggtgcccgacgcttgcaccgcttgcgacctggtgaacgtggacctggacgactgcatcttcgagcagtaataatctagagctccagcttttgttccc

SEQ ID NO: 3 - DNA, 1932 bp, adeno-associated virus 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 4 - DNA, 1915 bp, Artificial Sequence
  Synthetic construct
gggcgaattgggtaccatcgatatgcccggtttctacgagatcgtgatcaaggtgccctccgacctggacgagcacctgcccggtatctccgactccttcgtgaactgggtggccgagaaggagtgggagctgcctcccgactccgacatggacctgaacctgatcgagcaggctcccctgaccgtggctgagaagctgcagcgtgacttcctgaccgagtggcgtcgtgtgtccaaggctcccgaggctctgttcttcgtgcagttcgagaagggcgagtcctacttccacatgcacgtgctggtcgagaccaccggtgtcaagtccatggtgctgggccgtttcctcagccagatccgtgagaagctgatccagcgtatctaccgtggtatcgagcccaccctgcccaactggttcgctgtgaccaagacccgtaacggtgctggcggtggtaacaaggtggtggacgagtgctacatccccaactacctgctgcccaagacccagcccgagctgcagtgggcttggaccaacatggaacagtacctgtccgcttgcctgaacctcaccgagcgtaagcgtctggtggcccagcacctgacccacgtgtctcagacccaggaacagaacaaggagaaccagaaccccaactccgacgctcccgtgatccgttccaagacctccgctcgttacatggaactggtcggttggctggtcgacaagggtatcacctccgagaagcagtggatccaggaagatcaggcttcctacatctccttcaacgctgcttccaactcccgttcccagatcaaggctgctctggacaacgctggcaagatcatgtccctgaccaagactgctcccgactacctggtcggccagcagcccgtggaggacatctcctccaaccgcatctacaagatcctcgagctgaacggttacgacccccagtacgctgcctccgttttcctgggttgggctaccaagaagttcggcaagcgtaacaccatctggctgttcggtcccgctaccaccggcaagaccaacatcgctgaggctatcgctcacaccgtgcccttctacggttgcgtgaactggaccaacgagaacttccccttcaacgactgcgtggacaagatggtgatttggtgggaggaaggcaagatgaccgctaaggtggtcgagtccgctaaggctatcctgggcggttccaaggtccgcgtggaccagaagtgcaagtcctccgctcagatcgaccccacccccgtgatcgtgacctccaacaccaacatgtgcgctgtgatcgacggtaactccaccaccttcgagcaccagcagcctctgcaggaccgtatgttcaagttcgagctgacccgtcgtctggaccacgacttcggcaaggtgaccaagcaggaagtgaaggacttcttccgttgggctaaggaccacgtggtggaggtggagcacgagttctacgtgaagaagggtggcgctaagaagcgtcccgctccctccgacgctgacatctccgagcccaagcgtgtgcgcgagtccgtggcccagccctccacctccgacgccgaggcttccatcaactacgctgaccgctaccagaacaagtgctcccgtcacgtgggcatgaacctgatgctgttcccttgccgtcagtgcgagcgtatgaaccagaacagcaacatctgcttcacccacggccagaaggactgcctcgagtgcttccccgtgtccgagtcccagcccgtgtccgtggtgaagaaggcttaccagaagctctgctacatccaccacatcatgggcaaggtgcccgacgcttgcaccgcttgcgacctggtgaacgtggacctcgacgactgcatcttcgagcagtaataatctagagctccagcttttgttccc

SEQ ID NO: 5 - DNA, 2211 bp, Artificial Sequence
  Synthetic Construct
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagcgcttcaacgggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacgccaagtctgtcaatgtggactttactgtggacaataatggcgtgtattcagagcctcgccccattggcaccagatacctgactcgtaatctgtaa

SEQ ID NO: 6 - DNA, 2260 bp, Artificial Sequence
  Synthetic construct
gggcgaattgggtaccatcgatatggctgctgatggttacctgcccgactggctcgaggataccctgtccgagggtatccgtcagtggtggaagctgaagcccggtcccccccctcccaagcccgctgagaggcacaaggacgattcccgtggtctggtgctgcccggttacaagtacctgggccccttcaacggtctggacaagggcgagcccgtgaacgaggctgacgctgctgctctcgagcacgacaaggcttacgaccgtcagctggactccggtgacaacccctacctgaagtacaaccacgctgacgctgagttccaggaacgtctgaaggaggacacctccttcggcggtaacctgggtcgtgctgtgttccaggctaagaagcgtgttctcgagcccctgggtctggtggaggaacccgtcaagaccgctcccggcaagaagcgtcccgtcgagcactcccccgtggagcccgactcctcctccggcaccggcaaggctggccagcagcccgctcgtaagcgtctgaacttcggccagaccggtgacgctgactccgtgcccgacccccagcccctgggccagcctcccgctgctccctccggtctgggcaccaacaccatggctaccggttccggtgctcccatggctgacaacaacgagggtgctgacggtgtcggtaactcctccggtaactggcactgcgactccacctggatgggtgaccgtgtgatcaccacctccacccgtacctgggctctgcctacctacaacaaccacctgtacaagcagatctcctccgcttccaccggtgcttccaacgacaaccactacttcggttactccaccccctggggctacttcgacttcaaccgtttccactgccacttctccccccgtgactggcagcgtctgatcaacaacaactggggtttccgtcccaagaggctgaacttcaagctgttcaacatccaagtcaaggaggtcacccagaacgacggcaccaccaccatcgccaacaacctgacctccaccgtgcaggtgttcaccgactccgagtaccagctgccctacgtgctgggttccgctcaccagggttgcctgccccccttccccgctgacgtgttcatggtgccccagtacggctacctgaccctgaacaacggttcccaggctgtgggccgttcctccttctactgcctcgagtacttcccatcccagatgctgcgtaccggtaacaacttcaccttctcctacaccttcgaggacgtgcccttccactcctcctacgctcactcccagtccctggaccgtctgatgaaccccctgatcgaccagtacctgtactacctgtcccgtaccaacaccccttccggaaccaccacccagtcccgtctgcagttctcccaggctggtgcttccgacatccgtgaccagtcccgtaactggctgcccggtccctgctaccgtcagcaacgcgtgtccaagacctccgccgacaacaacaacagcgagtactcctggaccggtgctaccaagtaccacctgaacggtcgtgactccctggtgaaccccggtcccgctatggcttcccacaaggacgacgaggaaaagttcttcccccagtccggtgtcctgatcttcggcaagcagggttccgaaaagaccaacgtggacatcgagaaggtcatgatcaccgacgaggaagagatccgtaccaccaaccctgtggctaccgagcagtacggttccgtgtccaccaacctgcagcgtggtaaccgtcaagctgctaccgctgacgtcaacacccagggtgtcctgcccggcatggtctggcaggaccgtgacgtgtacctgcagggtcccatctgggctaagatcccccacaccgacggtcacttccacccctcccccctgatgggcggtttcggtctgaagcacccccctccccagatcctgatcaagaacacccccgtgcccgctaacccctccaccaccttctccgctgctaagttcgcttccttcatcacccagtactccaccggccaggtgtccgtggagatcgagtgggagctgcagaaggagaactccaagcgttggaaccccgagatccagtacacctccaactacgctaagtccgtgaacgtggacttcaccgtggacaacaacggtgtctactccgagccccgtcccatcggcacccgttacctgacccgcaacctgtaataatctagagctccagcttttgttccc

SEQ ID NO: 7 - DNA, 2217 bp, Artificial Sequence
  Synthetic construct
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 8 - DNA, 2266 bp, Artificial Sequence
  Synthetic construct
gggcgaattgggtaccatcgatatggctgctgacggttacctgcccgactggctcgaggataacctgtccgagggtatccgtgagtggtgggctctgaagcccggtgctcccaagcccaaggctaaccagcagaagcaggacgacggtcgcggtctggtgctgcccggttacaagtacctgggccccttcaacggtctggacaagggcgagcccgtgaacgctgctgacgctgccgctctcgagcacgacaaggcttacgaccagcagctgcaggctggtgacaacccctacctgcgttacaaccacgctgacgctgagttccaggaacgtctgcaggaagatacctccttcggcggtaacctgggtcgtgctgtgttccaggctaagaagcgtgtcctcgaacccctgggtctggtggaggaaggtgctaagaccgctcccggcaagaagcgtcccgtcgagccctccccccagcgttcccccgactcctccaccggtatcggcaagaagggccagcagcccgctcgtaagcgtctgaacttcggccagaccggtgactccgagtccgtgcccgacccccagcccctgggcgagcctcccgctgctccctccggtgtcggtcccaacaccatggccgctggcggtggtgctcccatggctgacaacaacgagggtgctgacggtgtcggttcctcctccggtaactggcactgcgactccacctggctgggtgaccgtgtgatcaccacctccacccgtacctgggctctgcctacctacaacaaccacctgtacaagcagatctccaacggcacctctggtggtgctaccaacgacaacacctacttcggttactccaccccctggggctacttcgacttcaaccgtttccactgccacttctccccccgtgactggcagcgtctgatcaacaacaactggggtttccgtcccaagcgcctgtccttcaagctgttcaacatccaagtcaaggaggtcacccagaacgagggcaccaagaccatcgctaacaacctgacctccactatccaggtgttcaccgactccgagtaccagctgccctacgtgctgggttccgctcaccagggttgcctgccccccttccccgctgacgtgttcatgatcccccagtacggctacctgaccctgaacaacggttcccaggctgtgggccgttcctccttctactgcctcgagtacttcccatcccagatgctgcgtaccggtaacaacttccagttcacctacaccttcgaggacgtgcccttccactcctcctacgctcactcccagtccctggaccgtctgatgaaccccctgatcgaccagtacctgtactacctgtcccgtacccagaccaccggtggcaccgctaacacccagaccctgggtttcagccagggtggccccaacactatggctaaccaggccaagaactggctgcccggtccctgctaccgtcagcaacgcgtgtccaccaccaccggccagaacaacaactccaacttcgcttggaccgctggcaccaagtaccacctgaacggtcgtaactccctggctaaccccggtatcgctatggctacccacaaggacgacgaggaacgtttcttcccctccaacggtatcctgatcttcggcaagcagaacgctgctcgtgacaacgctgactactccgacgtgatgctgacctccgaggaagagatcaagaccaccaaccccgtggctaccgaggaatacggtatcgtcgctgacaacctgcagcagcagaacaccgctccccagatcggcaccgtgaactcccagggtgctctgcccggcatggtctggcagaaccgtgacgtgtacctgcagggtcccatctgggctaagatcccccacaccgacggtaacttccacccctcccccctgatgggcggtttcggtctgaagcaccctcccccccagatcctgatcaagaacacccccgtgcccgctgacccccccaccaccttcaaccagtccaagctgaactccttcatcacccagtactccaccggacaggtgtccgtcgagatcgagtgggagctgcagaaggagaactccaagcgttggaaccccgagatccagtacacctccaactactacaagtccacctccgtggacttcgctgtgaacaccgagggcgtgtactccgagccccgtcccatcggcacccgttacctgacccgcaacctgtaataatctagagctccagcttttgttccc

SEQ ID NO: 9 - DNA, 2211 bp, Artificial Sequence
  Synthetic Construct
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 10 - DNA, 2260 bp, Artificial Sequence
  Synthetic Construct
gggcgaattgggtaccatcgatatggctgctgatggttacctgcccgactggctcgaggacaacctgtccgagggtatccgtgagtggtgggctctgaagcccggtgctccccagcccaaggctaaccagcagcaccaggacaacgctcgtggtctggtcctgcccggttacaagtacctgggtcccggtaacggtctggacaagggcgagcccgtgaacgctgctgacgctgccgctctcgagcacgacaaggcttacgaccagcagctgaaggctggtgacaacccctacctgaagtacaaccacgctgacgctgagttccaggaacgtctgaaggaggacacctccttcggcggtaacctgggtcgtgctgtgttccaggctaagaagcgtctgctcgagcccctgggtctggtggaggaagctgctaagaccgctcccggcaagaagcgtcccgtcgagcagtccccccaggaacccgactcctccgctggtatcggcaagtccggtgcccagcccgctaagaagaggctgaacttcggccagaccggtgacaccgagtccgtgcccgacccccagcccatcggcgagccccctgctgctccctccggtgtcggttccctgaccatggcttccggtggtggtgctcccgtggctgacaacaacgagggtgctgacggtgtcggctcctcctccggtaactggcactgcgactcccagtggctgggtgaccgtgtgatcaccacctccacccgtacctgggctctgcctacctacaacaaccacctgtacaagcagatctccaactccacctccggtggttcctccaacgacaacgcttacttcggttactccaccccctggggctacttcgacttcaaccgtttccactgccacttctccccccgtgactggcagcgtctgatcaacaacaactggggtttccgtcccaagcgtctgaacttcaagctgttcaacatccaagtcaaggaggtcaccgacaacaacggtgtcaagaccatcgctaacaacctgacctccaccgtgcaggtgttcaccgactccgactaccagctgccctacgtgctgggttccgctcacgagggttgcctgccccccttccccgctgacgtgttcatgatcccccagtacggctacctgaccctgaacgacggttcccaggctgtgggccgttcctccttctactgcctcgagtacttcccatcccagatgctgcgtaccggtaacaacttccagttctcctacgagttcgagaacgtgcccttccactcctcctacgctcactcccagtccctggaccgtctgatgaaccccctgatcgaccagtacctgtactacctgtccaagaccatcaacggttccggccagaaccagcagaccctgaagttctccgtggctggtccctccaacatggctgtgcagggtcgtaactacatccccggtccctcctaccgtcagcaacgcgtgtccaccaccgtgacccagaacaacaactccgagttcgcttggcccggtgcttcctcctgggccctgaacggtcgtaactccctcatgaaccccggtcccgctatggcttcccacaaggagggcgaggaccgtttcttccccctgtccggctccctgatcttcggcaagcagggcaccggtcgtgacaacgtggacgctgacaaggtcatgatcaccaacgaggaagagatcaagaccaccaaccccgtggctaccgagtcctacggccaggtggccaccaaccaccagtccgctcaggctcaggcccagaccggttgggtgcagaaccagggtatcctgcccggcatggtctggcaggaccgtgacgtgtacctgcagggtcccatctgggctaagatcccccacaccgacggtaacttccacccctcccccctgatgggcggtttcggcatgaagcacccccctccccagatcctgatcaagaacacccccgtgcccgctgacccccccaccgctttcaacaaggacaagctgaactccttcatcacccagtactccaccggccaggtgtccgtggagatcgagtgggagctgcagaaggagaactccaagcgttggaaccccgagatccagtacacctccaactactacaagtccaacaacgtggagttcgctgtgaacaccgagggcgtgtactccgagccccgtcccatcggcacccgttacctgacccgcaacctgtaataatctagagctccagcttttgttccc

SEQ ID NO: 11 - DNA, 2208 bp, Artificial Sequence
  Synthetic construct
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 12 - protein, 735 aa, Artificial Sequence [sequence omitted]

SEQ ID NO: 13 - protein, 736 aa, Artificial Sequence
  Synthetic Construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSASTGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYAKSVNVDFTVDNNGVYSEPRPIGTRYLTRNL